(19) United States
(12) Patent Application Publication
(10) Pub. No.: US 2025/0378903 A1
(43) Pub. Date: Dec. 11, 2025

(54) Techniques for Predicting the Effect of Mutations in Intrinsically Disordered Proteins (IDPs)

(71) Applicant: UNIVERSITY OF CENTRAL FLORIDA RESEARCH FOUNDATION, INC., Orlando, FL (US)

(72) Inventors: Aniket Bhattacharya, Orlando, FL (US); Swarnadeep Seth, Orlando, FL (US)

(21) Appl. No.: 19/232,158

(22) Filed: Jun. 9, 2025

Related U.S. Application Data
(60) Provisional application No. 63/657,206, filed on Jun. 7, 2024.

(51) Int. Cl. G16B 15/20; G16B 20/20; G16B 40/20

(52) U.S. Cl. CPC G16B 15/20; G16B 20/20; G16B 40/20

(57) ABSTRACT

Techniques diagnose an effect on a subject of a mutation in an intrinsically disordered protein (IDP), or intrinsically disordered region thereof, with a known value for gyration of the non-mutated IDP. Techniques include determining a quick value of gyration radius or end to end distance or both of the mutation based on output produced by inputting the values of a plurality of physical properties of the mutation to a neural network. The neural network is trained on a training set including multiple instances of training set values for gyration radius or end to end distance or both with corresponding training set values of the plurality of physical properties. Techniques include using a difference between the quick value and the known value to determine an effect of the mutation.

### BACKGROUND

Intrinsically Disordered Proteins (IDPs) or intrinsically disordered regions (IDRs) in a protein represent a significant portion of the human proteome and play crucial roles in progression of degenerative diseases such as Parkinson's disease, Alzheimer's disease, and Type II Diabetes. Unlike proteins those fold, IDPs lack well defined structures; their rapid conformational changes are hard to resolve experimentally often with conflicting results.

One of the significant challenges in the field is the identification of lethal mutations within IDPs, as these mutations are key to understanding disease mechanisms and developing targeted therapies. Traditional experimental and computational methods are impractical due to the structural flexibility of IDPs and the vast number of potential mutations. There is a need for a novel approach to rapidly identify lethal mutations, understand their structural implications, and develop potential therapies.

### SUMMARY OF INVENTION

Disclosed herein are embodiments to rapidly identify lethal mutations in IDPs, elucidate their structural consequences, and inform drug design for therapeutic interventions. This approach integrates machine learning (ML), polymer physics-based knowledge, advanced molecular dynamics simulation techniques, and chemistry-based structural specificity. The disclosed embodiments accelerate the mutation discovery process in IDPs by several orders of magnitude compared to current methods.

In a first set of embodiments, a method to diagnose an effect on a subject of a mutation in an intrinsically disordered protein or an intrinsically disordered region thereof, includes determining a mutation from a known amino acid sequence of an intrinsically disordered protein or an intrinsically disordered region thereof. The known amino acid sequence is associated with a known value of gyration radius or end-to-end distance or both. The method also includes determining values of a plurality of physical properties of the mutation based on inputting the mutation in the amino acid sequence into a polymer physics-based model. Furthermore, the method includes determining a quick value of gyration radius, or end-to-end distance, or both, of the mutation based on output produced by inputting the values of the plurality of physical properties of the mutation to a neural network. The neural network is trained on a training set including multiple instances of training set values for gyration radius or end-to-end distance or both with corresponding training set values of the plurality of physical properties. The method still further includes using a difference between the quick value and the known value to determine an effect of the mutation.

In some embodiments of the first set, the neural network comprises a plurality of fully connected hidden layers. In some of these embodiments, the neural network comprises six fully connected hidden layers; or each hidden layer is configured to drop a number of nodes by a factor of two or three, or alternate hidden layers alternate between a tanh activation function for the hidden layer and a RELU activation function for the hidden layer, or the first hidden layer following the input layer comprises 192 nodes, or the last hidden layer before the output layer uses a linear activation function, or some combination.

In some embodiments of the first set, the method even further includes performing detailed modeling to determine improved magnitude of gyration of mutations that have the determined effect greater than a first threshold. In some of these embodiments, the method yet further still includes performing drug screening for pathogenic mutations that have an improved magnitude greater than a second threshold.

In some embodiments of the first set, the plurality of physical properties includes five or more properties of a group including length (N), center of mass CM(m), center of charge CM(q), center of hydropathy CM(λ), mass mean field standard deviation (MFSTD), charge MFSTD, hydropathy MFSTD, entropy, charge entropy, net charge per residue (qnet=Q/N), net positive charge per residue (q+/N), net negative charge per residue (q−/N), charge asymmetry (charge decoration parameter), hydropathy asymmetry (hydropathy decoration parameter), contiguous patches of unit positive charge, contiguous patches of unit negative charge, contiguous patches of 0.5 positive charge, and contiguous patches of neutral charge.

In other sets of embodiments, an apparatus, computer-readable medium or system is configured to perform one or more steps of one or more of the above methods.

### BRIEF DESCRIPTION OF THE DRAWINGS

FIG. 1A is a block diagram that illustrates an example of a training set, according to an embodiment;

FIG. 1B is a block diagram that illustrates an example of a method for training a model by setting model parameters based on the training set, according to an embodiment;

FIG. 2A is a block diagram that illustrates an example of a neural network for illustration, according to an embodiment;

FIG. 2B is a plot that illustrates examples of activation functions used to combine inputs at any node of a feed forward neural network, according to various embodiments;

FIG. 3A and FIG. 3B are block diagrams that illustrate an example of the schematics of the computational (simulation+deep learning) workflow to predict the missense mutations of an example IDP which is then used for drug screening and clinical trial, according to an embodiment;

FIG. 3C is a flow chart that illustrates an example method to train and use a neural network for predicting gyration radius or length or some combination, according to an embodiment;

FIG. 4 is a block diagram that illustrates an example of a neural network structure, according to an embodiment;

FIG. 5A is a chart that illustrates an example of accuracy of the neural network in predicting gyrations of amino acid sequences, according to an example embodiment;

FIG. 5B is a plot that illustrates an example of accuracy of the neural network in predicting gyrations of mutations, according to an example embodiment;

FIG. 6 and FIG. 7 are charts that illustrate further examples of subsets of mutations with significant differences in gyrations from corresponding unmutated IDPs, according to two further example embodiments;

FIG. 8 is a block diagram that illustrates a computer system upon which an embodiment of the invention may be implemented; and

FIG. 9 illustrates a chip set upon which an embodiment of the invention may be implemented.

### DETAILED DESCRIPTION

Notwithstanding that the numerical ranges and parameters setting forth the broad scope are approximations, the numerical values set forth in specific non-limiting examples are reported as precisely as possible. Any numerical value, however, inherently contains certain errors necessarily resulting from the standard deviation found in their respective testing measurements at the time of this writing. Furthermore, unless otherwise clear from the context, a numerical value presented herein has an implied precision given by the least significant digit. Thus, a value 1.1 implies a value from 1.05 to 1.15. The term “about” is used to indicate a broader range centered on the given value, and unless otherwise clear from the context implies a broader range around the least significant digit, such as “about 1.1” implies a range from 1.0 to 1.2. If the least significant digit is unclear, then the term “about” implies a factor of two, e.g., “about X” implies a value in the range from 0.5× to 2×, for example, about 100 implies a value in a range from 50 to 200. Moreover, all ranges disclosed herein are to be understood to encompass any and all sub-ranges subsumed therein. For example, a range of “less than 10” for a positive only parameter can include any and all sub-ranges between (and including) the minimum value of zero and the maximum value of 10, that is, any and all sub-ranges having a minimum value of equal to or greater than zero and a maximum value of equal to or less than 10, e.g., 1 to 4.

### Machine Learning to Generate an IDP Gyration Predictive Model

Effective training of a machine learning system with the characteristics described above can be achieved using neural networks, widely used in image processing and natural language processing.

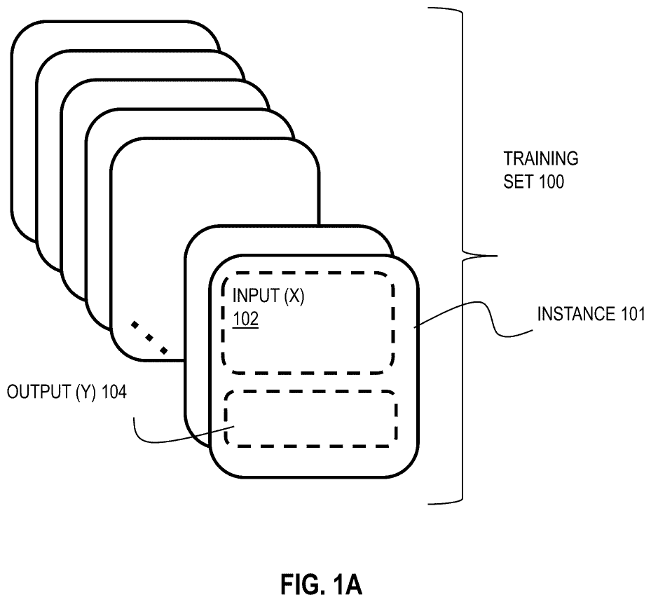

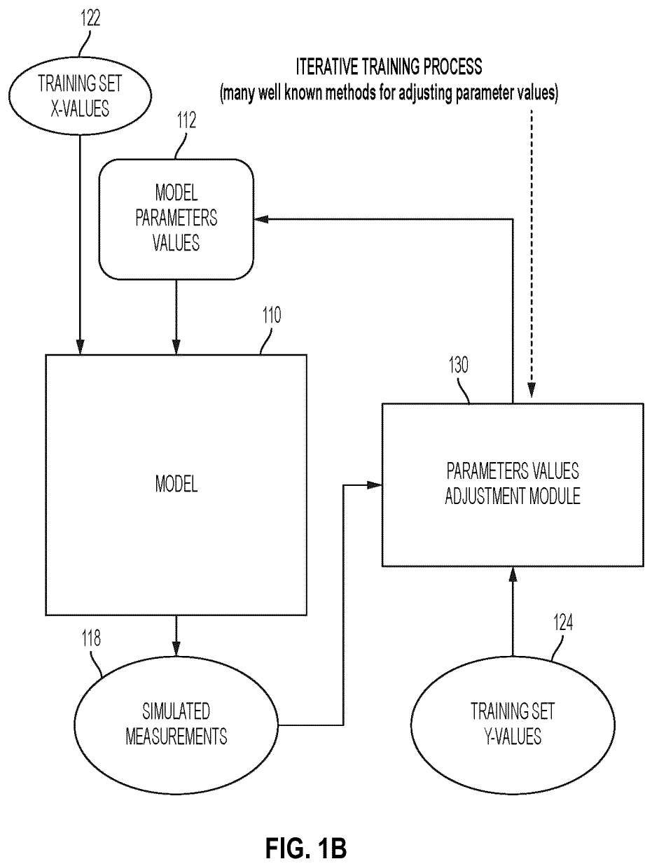

FIG. 1A is a block diagram that illustrates an example training set 100, according to an embodiment. The training set 100 includes multiple instances, such as instance 101. The instances 101 for the set 100 are selected to be appropriate for a particular use. Each training set 100 instance 101 includes input data 102 (represented by the variable X, such as values for one or more input properties expected to be relevant to a desired output) and output data 104 (represented by variable Y, such as a value for a gyration parameter) desired to be output from the artificial intelligence machine given the input data X 102.

In general, an artificial intelligence machine is programmed with a model M that includes a variety of adjustable parameters P, the values for which are determined by training with the training set 100 to provide a given output 104 for a given input 102 of each instance 101 of the training set 100. Many training methods are known and can be used alone or in combination to train the machine model based on the training set 100.

During machine learning, a model M is selected appropriate for the purpose and data at hand. One or more of the model M adjustable parameters P is uncertain for that particular purpose and the values for such one or more parameters are learned automatically. Innovation is often employed in determining which model to use and which of its parameters to fix and which to learn automatically. The learning process is typically iterative and begins with an initial value for each of the uncertain parameters P and adjusts those prior values based on some measure of goodness of fit of its Model output YM with known results Y for a given set of values for input context variables X from an instance 101 of the training set 100.

FIG. 1B is a block diagram that illustrates an example automatic process for learning values for uncertain parameters P 112 of a chosen model M 110. The model M 110 can be a Boolean model for a result Y of one or more binary values, each represented by a 0 or 1 (e.g., representing FALSE or TRUE respectively), a classification model for membership in two or more classes (either known classes or self-discovered classes using cluster analysis), other statistical models (such as mean and standard deviation of a Gaussian or Poisson function, shape and scale of a Gamma function, multivariate regression, or neural networks), or a physical model, or some combination of two or more such models. A physical model differs from the other purely data-driven models because a physical model depends on mathematical expressions for known or hypothesized relationships among physical phenomena. When used with machine learning, the physical model includes one or more parameterized constants, such as propagation loss coefficients, that are not known or not known precisely enough for the given purpose.

During training depicted in FIG. 1B, the model 110 is operated with current values 112 of the parameters P, including one or more uncertain parameters of P (initially set arbitrarily or based on order of magnitude estimates) and values of the input variables X 102 from an instance 101 of the training set 100. The values 116 of the output YM from the model M, also called simulated measurements, are then compared to the values 124 of the known or desired result variables Y 104 from the corresponding instance 101 of the training set 100 in the parameters values adjustment module 130.

The parameters values adjustment module 130 implements one or more known or novel procedures, or some combination, for adjusting the values 112 of the one or more uncertain parameters of P based on the difference between the values of YM and the values of Y 104. The difference between YM and Y 104 can be evaluated using any known or novel method for characterizing a difference, including least squared error, maximum entropy, fit to a particular probability density function (pdf) for the errors, e.g., using a priori or a posterior probability. The model M 110 is then run again with the updated values 112 of the uncertain parameters of P and the values of the context variables X 102 from a different instance 101 of the training set 100. The updated values 116 of the output YM from the model M110 are then compared to the values of the known result variables Y 102 from the corresponding instance 101 of the training set 100 in the next iteration of the parameter values adjustment module 130.

The process of FIG. 1B continues to iterate until some stop condition is satisfied. Many different stop conditions can be used. The model can be trained by cycling through all or a substantial portion of the training set. In some embodiments, a minority portion of the training set 200 is held back as a validation set. The validation set is not used during training, but rather is used after training to test how well the trained model works on instances that were not included in the training. The performance on the validation set instances, if truly randomly withheld from the instances used in training, is expected to provide an estimate of the performance of the trained model in producing YM when operating on target data X with results Y that are not already known. Typical stop conditions include one or more of a certain number of iterations, a certain number of cycles through the training portion of the training set, producing differences between YM and Y less than some target threshold, producing successive iterations with no substantial reduction in differences between YM and Y 104, and errors in the validation set less than some target threshold, or no substantial differences in the parameter values P on successive iterations, among others, or some combination.

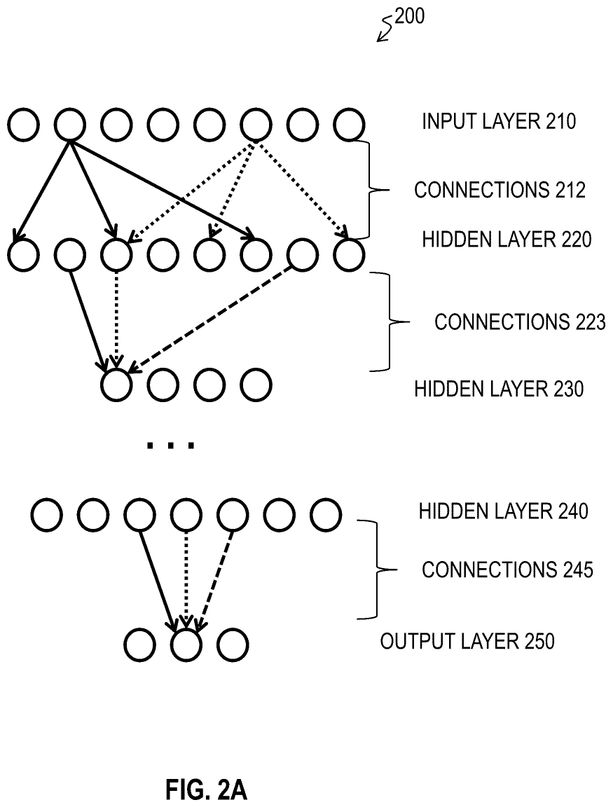

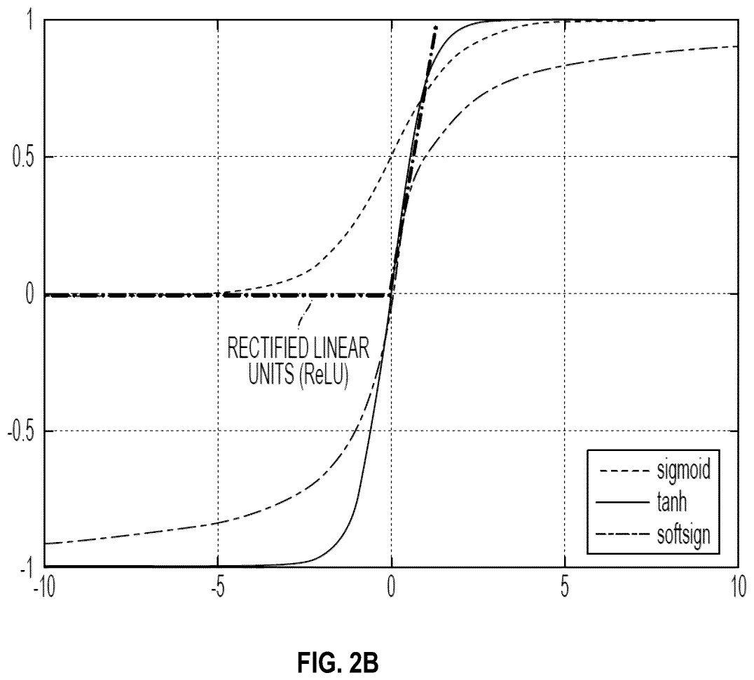

FIG. 2A is a block diagram that illustrates an example neural network 200 for illustration used as a model M or portion thereof in some embodiments. A neural network 200 is a computational system, implemented on a general-purpose computer, or field programmable gate array, or some application specific integrated circuit (ASIC), or some neural network development platform, or specific neural network hardware, or some combination. The neural network is made up of an input layer 210 of nodes, at least one hidden layer 220, 230 or 240 of nodes, and an output layer 250 of one or more nodes. Each node is an element, such as a register or memory location, that holds data that indicates a value. The value can be code, binary, integer, floating point, or any other means of representing data. Values in nodes in each successive layer after the input layer in the direction toward the output layer is based on the values of one or more nodes in the previous layer. The nodes in one layer that contribute to the next layer are said to be connected to the node in the later layer. Connections 212, 223, 245 are depicted in FIG. 2 as arrows. The values of the connected nodes are combined at the node in the later layer by summation, multiplication, convolution or other operation using weights and is then filtered using some activation function with scale and bias (additional weights) that can be different for each connection. Neural networks are so named because they are modeled after the way neuron cells are connected in biological systems. A fully connected neural network (FCNN) has every node at each layer connected to every node at any previous or later layer.

FIG. 2B is a plot that illustrates example activation functions used to combine inputs at any node of a neural network. These activation functions are normalized to have a magnitude of 1 and a bias of zero; but when associated with any connection can have a variable magnitude given by a weight and centered on a different value given by a bias. The values in the output layer 350 depend on the values in the input layer and the activation functions used at each node and the weights and biases associated with each connection that terminates on that node. The sigmoid activation function (dashed trace) has the properties that values much less than the center value do not contribute to the combination (a so called switch-off effect) and large values do not contribute more than the maximum value to the combination (a so called saturation effect), both properties frequently observed in natural neurons. The tanh activation function (solid trace) has similar properties but allows both positive and negative contributions. The softsign activation function (short dash-dot trace) is similar to the tanh function but has much more gradual switch and saturation responses. The rectified linear units (ReLU) activation function (long dash-dot trace) simply ignores negative combinations from nodes on the previous layer; but, increases linearly with positive combinations from the nodes on the previous layer; thus, ReLU activation exhibits switching but does not exhibit saturation. The identity activation function applies identity operation on input data so output data is proportional to the input data; thus, it exhibits neither switching nor saturation effects. In some embodiments, the activation function operates on individual connections before a subsequent operation, such as summation or multiplication; in other embodiments, the activation function operates on the sum or product of the values in the connected nodes. In other embodiments, other activation functions are used, such as kernel convolution.

An advantage of neural networks is that they can be trained to produce a desired output from a given input without knowledge of how the desired output is computed. There are various algorithms known in the art to train the neural network on example inputs with known outputs. Typically, the activation function for each node or layer of nodes is predetermined, and the training determines the weights and biases for each connection. A trained network that provides useful results, e.g., with demonstrated good performance for known results, is then used in operation on new input data not used to train or validate the network.

In some neural networks, the activation functions, weights and biases are shared for an entire layer. This provides the trained network with shift and rotation invariant responses. The hidden layers can also consist of convolutional layers, pooling layers, fully connected layers, and normalization layers. The convolutional layer has parameters made up of a set of learnable filters (or kernels), which have a small receptive field. In a pooling layer, the activation functions perform a form of non-linear down-sampling, e.g., producing one node with a single value to represent several nodes in a previous layer. There are several non-linear functions to implement pooling among which max pooling is the most common. A normalization layer simply rescales the values in a layer to lie between a predetermined minimum value and maximum value, e.g., 0 and 1, respectively.

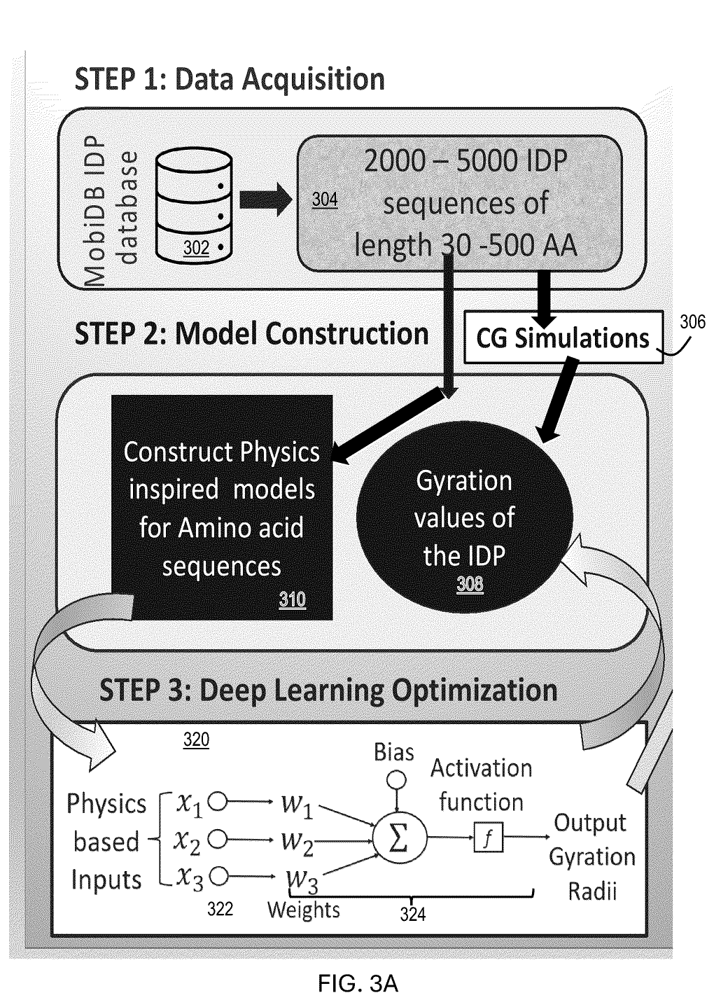

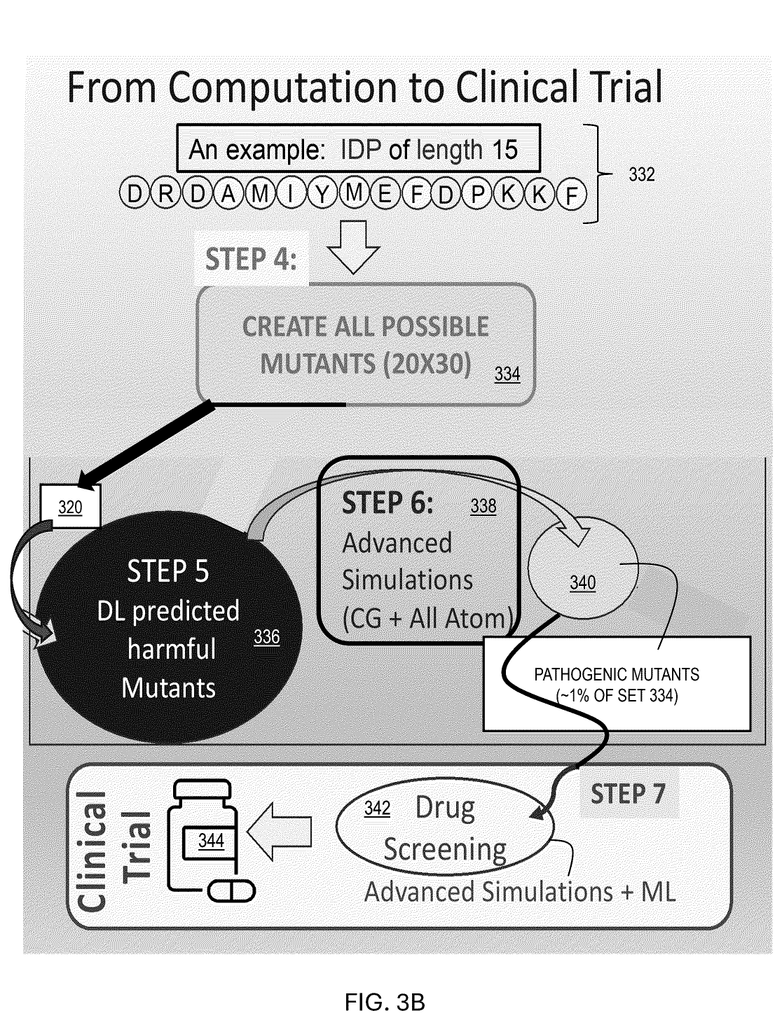

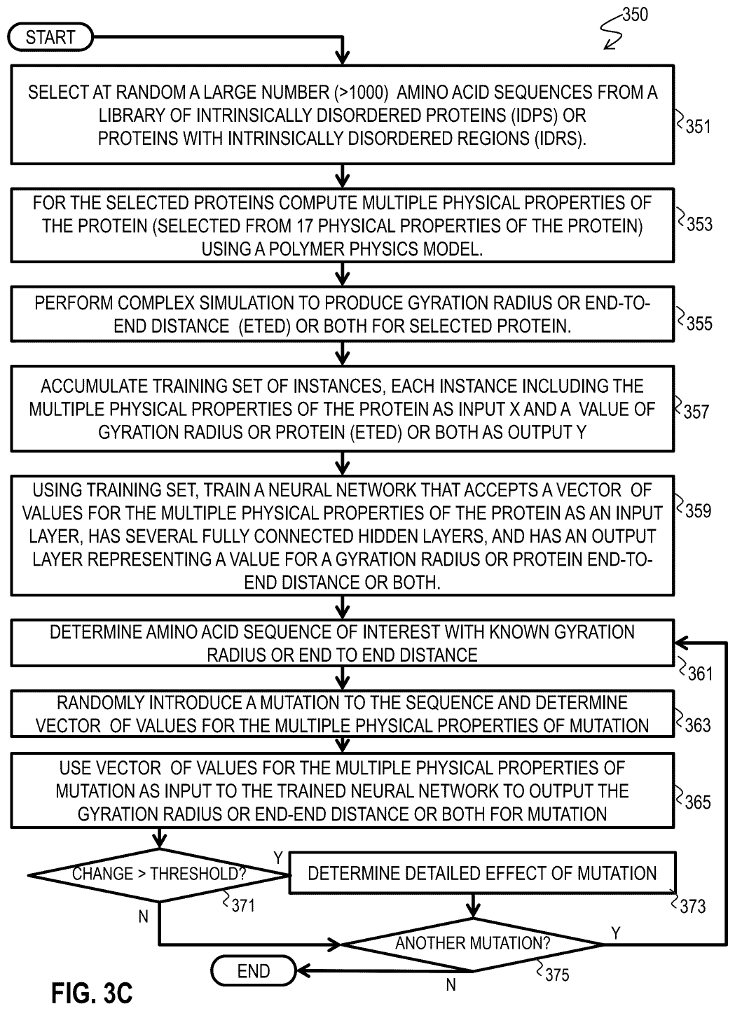

FIG. 3A and FIG. 3B are block diagrams that illustrate an example of the schematics of the computational (simulation+deep learning) workflow to predict the missense mutations of an example IDP which is or can be used for drug screening and clinical trials

Generate Training Sets for Deep Learning. This process develops training sets by combining polymer physics-based feature sets of IDPs with simulation results from a modest number (˜1,000 to 10,000) of random IDPs from a curated database, such as MobiDB IDP.

In Step 1 depicted in FIG. 3A, the curated database such as MobiDB IDP is accessed. Also available for a training set are the data provided by Sickmeier, M. et al. DisProt: the Database of Disordered Proteins. Nucleic Acids Research 35, D786-D793 (2007) and a database of intrinsically disordered proteins available at subdomain mobidb of subdomain bio of domain unipd of superdomain it. Such databases are represented in FIG. 3A by IDP database 302. Note that there are 22 distinct amino acids that are assembled in an amino acid sequence to form proteins, including all IDPs.

Of the amino acid (AA) sequences for IDPs in database 302, a manageable portion is selected for a data structure 304 used to generate of a training set. In example embodiments, the AA sequences of length 30 to 500 amino acids for any number of IDPs, e.g., from about 1000 IDPs up to about 5000 IDPs are considered a manageable number of IDPs for generating a training set, such as the 2000 up to 500 used for data structure 304. Each IDP in data structure 304 is used to generate one instance, e.g. 101, for a training set, e.g. 100.

In Step 2 depicted in FIG. 3A, the physics-inspired features used as input, e.g., X 102, for each instance, e.g., 101, in a training set, e.g., 100, for machine learning are obtained directly from each amino acid sequence itself in data structure 304 and do not require any simulation run. Example physics inspired features are described in more detail below, but include properties for which values indicate length and mass and charge distributions along stretches of an AA chain which are unfolded and can thus gyrate more freely. In some embodiments, the input values X 102 for each instance 101 are stored in data structure 310 for all instances in training set 100.

Note that an advantage of using the physical properties of the sequence is that they are constant in number regardless of the actual number of amino acids in the sequence. Thus, a single neural network can be used to predict the gyrations of an amino acid sequence of any length. This realization to reduce the input layer to a constant set of these physical properties instead of the actual sequence is a major advance represented by the approach presented here.

The desired output, e.g., Y 104, for each instance, e.g. 101, is a value of a radius of the gyration or a value of an end-to-end distance, or both. These instance 101 output 104 values are obtained from a coarse-grained (CG) simulation performed by module 306 based on the AA sequences for each IDP in data structure 304. This simulation is extremely complicated and time consuming, involving days of computations on high-end computers. Thus, performing these complex computations a limited number of times to train a neural network to produce quick estimates of the gyration radius of a new protein is advantageous.

The simulated values of gyration radius or end-to-end distance, or both, is stored in data structure 308 for all instances in training set 100. Data structures 310 and 308 serve as the foundation inputs X and outputs Y of for all instances 101 of training set 100 for machine/deep learning algorithms.

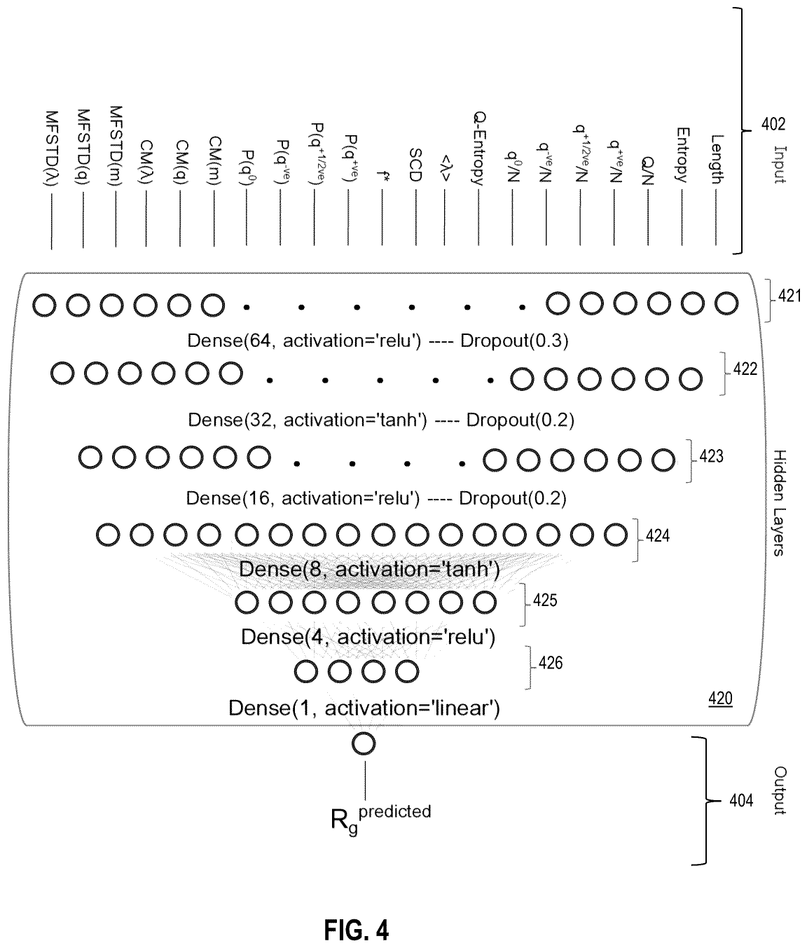

Perform Deep Learning. In Step 3 depicted in FIG. 3A, a machine learning model is trained using the training set established during step 2 described above. In the illustrated embodiment, the machine learning model is a neural network 320. When the machine learning model is a neural network, the training is called “deep learning.” The neural network 320 is illustrated schematically by showing one example node in a first hidden layer. In the illustrated schematic, values of three physics-inspired properties are indicated by input values x1, x2, x3 at corresponding nodes of an input layer 322. These nodes are connected to a node 324 in a first hidden layer. At that node 324, each of these values x1, x2, x3 is multiplied by a corresponding weight w1, w2, w3, respectively, and summed in a module indicated by a summation sign (Σ), which sum is offset by a Bias for the layer 324 and then filtered by an activation function ƒ for the node 324. The output is passed to the connected nodes of the next hidden layer (not shown) until a single output node indicates a value for the gyration radius. The parameters of the neural network 320 that are adjusted during training include the weights w1, w2, w3 for each connected node in each layer. Typically, the combining function, such as summation, the bias, and the activation function are predetermined for each node. Often, the combining function, bias and activation function are constant for an entire layer. In an example embodiment depicted in FIG. 4, described below, all nodes in each layer are fully connected to the nodes in the preceding layer, i.e., each node in the preceding layer is connected by a separate weight w to each node in the next layer. In the example embodiment, each node in one layer uses the same combination function, bias and activation function. In various embodiments, the combination, bias and or activation function may vary from one layer to the next. During step 3 the weights, e.g., w1, w2, w3, for every connection between every layer are adjusted so that the given input X 102 in data structure 310 provides a close approximation of the given output Y 104 in data structure 308 for the instances 101 of training set 100 based on the AA sequences in data structure 304.

Use trained NN to predict the anomalous gyration radii. This deep learning network is then used, as depicted in FIG. 3B, to identify potential harmful mutations by comparing the change of the gyration radius of each potential mutant output by the neural network with the known gyration radius of the wild-type sequence in the original database 302.

For example, an IDP of interest 332 is selected. The example IDP has a given sequence of 15 amino acids. Each of the 20 unique amino acids is indicated by one of 20 letters selected from the 26 letter English alphabet. For the purposes of the examples presented here, it is not necessary to know which letter refers to which amino acid, only that a known amino acid is represented by a letter and that known amino acid has known molecular structure and thus known values for various electrical and mechanical properties.

Of concern are the few mutations from this known IDP that greatly affect the folding and gyration properties of the mutated protein from the known IDP—the latter also called the wild type IDP. The mutations of concern potentially are capable of inducing functional impairment and, thus, driving disease progression in IDPs. To find these few mutations of concern, in step 4 all possible mutations are formed of the wild type IDP. Thus, for each of the 15 amino acids there are 19 possible AA replacements. Including the wild type, there are 15×20 different 15 amino acid sequences to be explored for gyration radius or end-to-end distance. In step 5 the trained neural network 320 is run on all possible mutations, giving a quick value for gyration radius, or end-to-end distance, or both, for each mutation. Of these quick results, the values of gyration radius, or end-to-end distance, for many mutations do not differ much from the known gyration radius, or end-to-end distance, of the wild type. Such mutations with small differences are not of great concern. Step 5 includes, identifying those mutations with a potential to be of concern. Those mutations are mutations with a quick gyration radius, or end-to-end-distance, difference greater than a threshold difference in either a positive or negative direction. Such mutations with greater than a threshold difference from the wild type are stored in data structure 336 as deep learning predicted harmful mutants. Thus, this includes determining mutations that have an effect greater than a first threshold.

The quick results from the trained neural network narrows down the search space from tens of thousands of mutants in data structure 334 to only tens of mutant sequences in data structure 336.

Compare Dynamic Conformations. Then, in step 6, explicit complex computational simulations are run using module 338 on those selected few mutant sequences in data structure 336 with differences greater than the threshold which are perceived as potentially capable of inducing functional impairment and, thus, driving disease progression in IDPs. A meticulous comparison of dynamic conformations between a healthy IDP and its mutant counterparts is thus conducted in module 338, allowing for a better understanding of the structural implications of mutations. The results are identification of pathogenic mutants stored in data structure 340. Thus, this includes performing detailed modeling to determine improved magnitude of gyration and improved differences from the wild type. It is expected that this list of pathogenic mutants is only about 1% or less of the possible mutations stored in data structure 334.

In step 7, the identified pathogenic mutants in data structure 340 are used in drug screening in module 342 to identify drugs 244 that potentially can offset the effects of the large differences in IDP gyration. Such drugs are suitable for clinical trials.

Method details. FIG. 3C is a flow chart that illustrates an example method to train and use a neural network for predicting gyration diameter or length or some combination, according to an embodiment. Although steps are depicted in FIG. 3C as integral steps in a particular order for purposes of illustration, in other embodiments, one or more steps, or portions thereof, are performed in a different order, or overlapping in time, in series or in parallel, or are omitted, or one or more additional steps are added, or the method is changed in some combination of ways.

In step 351, a large number (>1000) of amino acid sequences are selected at random from a library of intrinsically disordered proteins (IDPs) or proteins with intrinsically disordered regions (IDRs). For example, between 1000 and 5000 amino acid sequences for IDPs or IDRs or some combination are selected from a library of known IDPs or IDRs or both, such from databases listed above The library in some embodiments includes information about gyration radius [symbol] R_{g} [symbol] or end-to-end distance [symbol] R_{N} [symbol] . for one or more of the selected IDPs or IDRs or both.

In step 353, for the selected proteins, multiple physical properties of each protein is computed using a polymer physics-based model known in the art. For example the polymer physics-based model is described in Dignon, G. L., Zheng, W., Kim, Y. C., Best, R. B. & Mittal, J. Sequence determinants of protein phase behavior from a coarse-grained model. PLOS Computational Biology 14, e1005941 (2018), and the physical properties are selected from 21 physical properties of the protein that are output by this model including those in Table 1, and described in more detail below.

TABLE 1
Input candidates.

Length _{——}_{——}_{——}_{——}
Entropy _{——}_{——}_{——}_{——}
Q/N _{——}_{——}_{——}_{——}
q^{+ve}/N _{——}_{——}_{——}_{——}
q^{+1/2ve}/N _{——}_{——}_{——}_{——}
q^{-ve}/N _{——}_{——}_{——}_{——}
q⁰/N _{——}_{——}_{——}_{——}
Q-Entropy _{——}_{——}_{——}_{——}
<λ> _{——}_{——}_{——}_{——}
SCD _{——}_{——}_{——}_{——}
f* _{——}_{——}_{——}_{——}
P(q^{+ve}) _{——}_{——}_{——}_{——}
P(q^{+1/2ve}) _{——}_{——}_{——}_{——}
P(q^{-ve}) _{——}_{——}_{——}_{——}
P(q⁰) _{——}_{——}_{——}_{——}
CM(m) _{——}_{——}_{——}_{——}
CM(q) _{——}_{——}_{——}_{——}
CM(λ) _{——}_{——}_{——}_{——}
MFSTD(m) _{——}_{——}_{——}_{——}
MFSTD(q) _{——}_{——}_{——}_{——}
MFSTD(λ) _{——}_{——}_{——}_{——}

These parameters are based on the amino acid sequence as described in the following paragraphs.

IDPs are mostly polyampholytes and polyelectrolytes. In addition, each amino acid bead has different mass and hydropathy indices. The hydrophobic or hydrophilic character of an amino acid is its hydropathic character, hydropathicity, or hydropathy. Therefore, it is expected that the length of the chain and the distribution of the positive and negative charge residues, as well as their hydropathy values, will affect the conformational properties. This justifies inclusion of the IDP sequence length (N), the center of mass of the IDP sequence CM(m), center of the charge CM(q), and center of the hydropathy parameter CM(λ). The center of mass is calculated as

$$CM ( m ) = \frac{1}{M} {\sum }_{k}^{N} \frac{i - k}{k} {m}_{i} , \qquad ( 1 )$$

where, mᵢ is the mass of the ith amino acid, M is the total mass of all amino acids. The value of k is either N/2 or (N−1)/2 depending upon the even and odd number of residues. Under this construction, the range of CM(i) can vary between (−1, 1) that captures the asymmetry in the residue mass distribution. Due to the defined bounding range of CM(i), this metric can be applied to characterize asymmetry in sequence distribution regardless of the IDP length.

Similarly, we construct the CM(q) and CM(λ) by replacing i by corresponding charge and hydropathy sequence. Unlike the mass or hydropathy scale which can take only positive values, we have both negative and positive residues. Hence, the modified expression for CM(q) can be written as

$$CM ( q ) = \frac{1}{{\sum }_{i}^{N} \vert {q}_{i} \vert} {\sum }_{i}^{N} \frac{i - k}{k} {q}_{i} . \qquad ( 2 )$$

Along with the center of the sequence distribution, we design a new metric that captures the fluctuation in the sequence space. The mean field standard deviation (MFSTD) metric subtracts the mean values of the sequence property from the sequence to effectively calculate the standard deviation. For a sequence of mass we calculate

$$MFSTD ( m ) = \sqrt{\frac{1}{M} {\sum }_{i}^{N} {( {m}_{i} - m \text{?} )}^{2}} \text{?} \qquad ( 3 )$$

$$\text{?} \text{indicates text missing or illegible when filed}$$

wherein

$$\text{?} m \text{?} = \frac{1}{N} {\sum }_{i}^{N} {m}_{i} \text{?} .$$

$$\text{?} \text{indicates text missing or illegible when filed}$$

This formula is applied to calculate MFSTD(q) and MFSTD(λ) with the charge and hydropathy sequence, respectively.

Entropy in the sequence space plays an essential role to capture the sequence dependent properties of the IDPs. We calculate Shannon entropy of the FASTA sequence as well as the charge sequence of the IDPSs as

$$Entropy ( i ) = - {\sum }_{i} - {p}_{i} {log}_{2} ( {p}_{i} ) . \qquad ( 4 )$$

The variable i represents sequence indices. For each unique character i, nᵢ represents the count of occurrences of that character in the sequence. The probability pᵢ of character i occurring in the sequence is calculated as the ratio of nᵢ to the total sequence length N. In the similar logic, we calculate the charge entropy Q-Entropy using the charge sequence in place of the FASTA sequence.

The net charge par residue (qnet=Q/N), net positive, and negative charges par residue q+/N and q−/N are considered as general inputs. The effect of the hydropathy parameters is included in the average hydropathy [symbol] λ [symbol] and the center of mass. Das and Pappu introduced charge asymmetry parameter

$${f}^{⋆} = \frac{{( {f}^{+} - {f}^{-} )}^{2}}{{f}^{+} + {f}^{-}} ,$$

where ƒ⁺/ƒ⁻ is the net positive/negative charge per residue of an ID, which is an important characteristics of the IDPs. We calculate the e charge decoration parameter

$$S C D = \frac{1}{N} {\sum }_{i = 2}^{N} {\sum }_{j = 1}^{i - 1} {q}_{i} {q}_{j} \cdot {\vert i − j \vert}^{0.5} \qquad ( 5 )$$

and in a similar construction is used to calculate the SHD from the hydropathy values of the amino acids.

The functions

$$P ( {q}^{+ ve} ) , P ( {q}^{+ \frac{1}{2} ( + ve )} ) ,$$

and P (q^{-ve} identify contiguous patches of the specified charge types within the charge sequence. These functions iterate through the sequence, counting consecutive occurrences of charge types, and append the counts to frequency of the charge patch. Any patch with a count of 1 is removed from patch frequency count. Finally, the functions return the maximum counts of consecutive occurrences of individual charge types as the most frequent patch length. If no patches are found, the functions return 0.
q^{+ve} is the fraction of unit positive charges per IDP
q^{-ve} is the fraction of unit negative charges per IDP
q^{+(1/2)ve} is the fraction of 0.5 positive charges per IDP
Here the unit charge is measured in units of charge of an electron or a proton.
q⁰ is the fraction of neutral amino acid beads in the IDP

We use some or all of these twenty-one important linearly independent and uncorrelated physics-based features derived from the IDP sequence information as input vector to train the MLP network as described in the next section. To summarize the preceding material, the twenty-one features of an amino acid (AA) sequence are {1} length (N), {2} center of mass CM(m), {3} center of the charge CM(q), {4} center of hydropathy CM(λ), {5} mass mean field standard deviation (MFSTD(m)), {6} charge MFSTD(q), {7} hydropathy MFSTD(λ), {8} entropy, {9} charge entropy, {10} net charge par residue (qnet=Q/N), {11} net positive charge par residue q^{+ve}/N, {12} net half positive charge par residue q^{+1/2ve}/N, {13} net negative charge par residue q^{-ve}/N, {14} net neutral charge par residue q⁰/N, {15} charge asymmetry ƒ*, {16} charge decoration parameter (SCD), {17} hydropathy asymmetry (<λ>), {18} contiguous patches of unit positive charge (Pq^{+ve}), {19} contiguous patches of unit negative charge (Pq^{-ve}), {20} contiguous patches of 0.5 positive charge (Pq^{1/2ve}), and {21} contiguous patches of neutral charge (Pq⁰). Using a constant few, or all, of these physics-based properties to train a neural network means that the trained neural network can be used for amino acid sequences of any length.

In step 355, a time-consuming complex simulation is performed to produce gyration radius or end-to-end distance for a selected protein in library for which those outputs are not already known. As IDPs are flexible, they can explore a large number of configurations and simulation is therefore needed for approximately 10 Rouse relaxation time to obtain the correct radius of gyration and other conformational properties. Thus, equilibration followed by taking statistics over several thousand uncorrelated conformations may take 2-3 days CPU wall time in the UCF STOKES CPU cluster. This step is so time consuming that it is impractical to do it for millions of potential mutations that are to be explored. But it is practical to perform once for just the few thousand selected proteins. In some embodiments all the selected proteins have known gyration radius (R_{g}) or end-to-end distance [symbol] R_{N} [symbol] or both; and step 355 can be omitted for some or all of the training set.

In step 357, a training set of instances is accumulated. Each instance includes two or more of the multiple physical properties of the protein as input X and a value of gyration radius [symbol] R_{g} [symbol] or protein [symbol] R_{N} [symbol] , or both, as output Y. In some embodiments, all the physical properties output by the polymer physics-based model in step 353 are used.

In some embodiments of step 357, statistical studies are performed to deduce a subset of the physical properties that best explain variations in [symbol] R_{g} [symbol] or [symbol] R_{N} [symbol] , or both, and only these properties are included in the training set as inputs X. Such statistical studies include principal component analysis, linear regression, among others. For example, extra tree regressors fit a number of randomized decision trees (extra-trees) on various sub-samples of the dataset and uses averaging to improve the predictive accuracy and to limit over-fitting. Thus, in an example embodiment, using Extra Tree Regressors, five of the most significant contributors to gyration radius or end-to-end distance include in order of decreasing significance length (N), center of hydropathy (CM(λ)) sequence charge decoration (SCD), fractional charge asymmetry ƒ* and center of mass (CM(m)).

In step 359, using the training set assembled in step 357, a neural network is trained. The neural network accepts a vector of values for the multiple physical properties of the protein as an input layer, has several fully connected hidden layers, and has an output layer representing a value for a gyration radius [symbol] R_{g} [symbol] or protein end-to-end distance [symbol] R_{N} [symbol] , or both. In one embodiment, depicted in FIG. 4, a particular number of fully connected hidden layers with particular activation functions are set and trained using the training data. But in other embodiments, other numbers of layers, with varying number of nodes in each layer, including one or more pooling layers or normalizing layers are used. In some embodiments, hyperparameter tuning is used to obtain an advantageous, even optimal, number of neurons for better and faster performance.

FIG. 4 is a block diagram that illustrates an example of a neural network structure, according to an embodiment. The weights are trained for the hidden layers, with the stated number of fully connected nodes, and stated activation functions, for this particular structure, according to this embodiment to output [symbol] R_{g} [symbol] . It is noted that, in the example neural network, the first hidden layer has many more nodes (e.g., 192) than in the input layer (e.g., 21). This is advantageous in discovering unique combinations of input properties that constructively combine to predict gyration output. This specific architecture provided acceptable performance. In other embodiments, other architectures that give the same or better performance may be used.

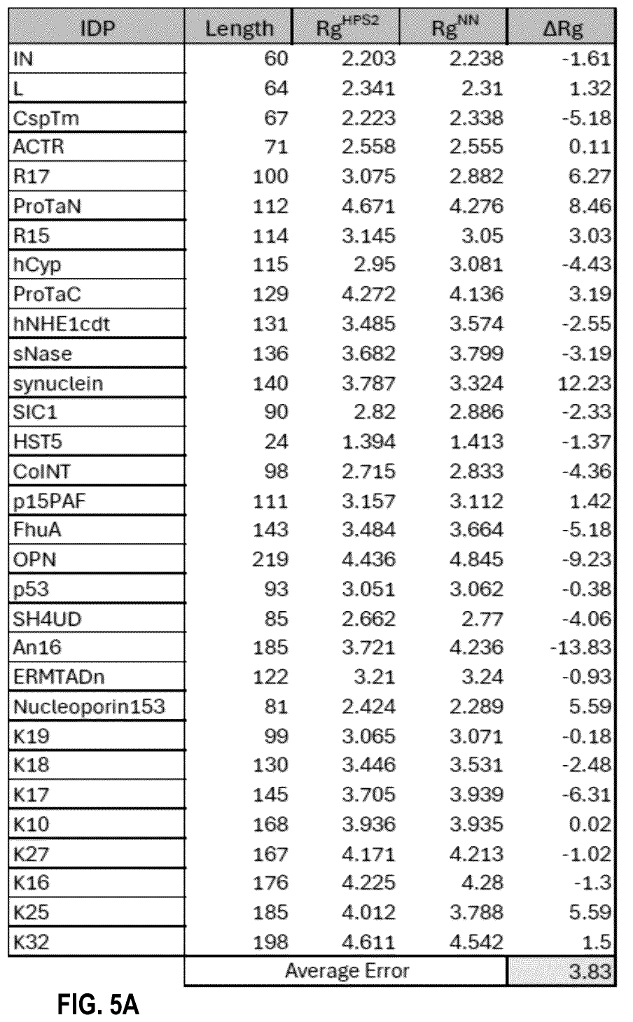

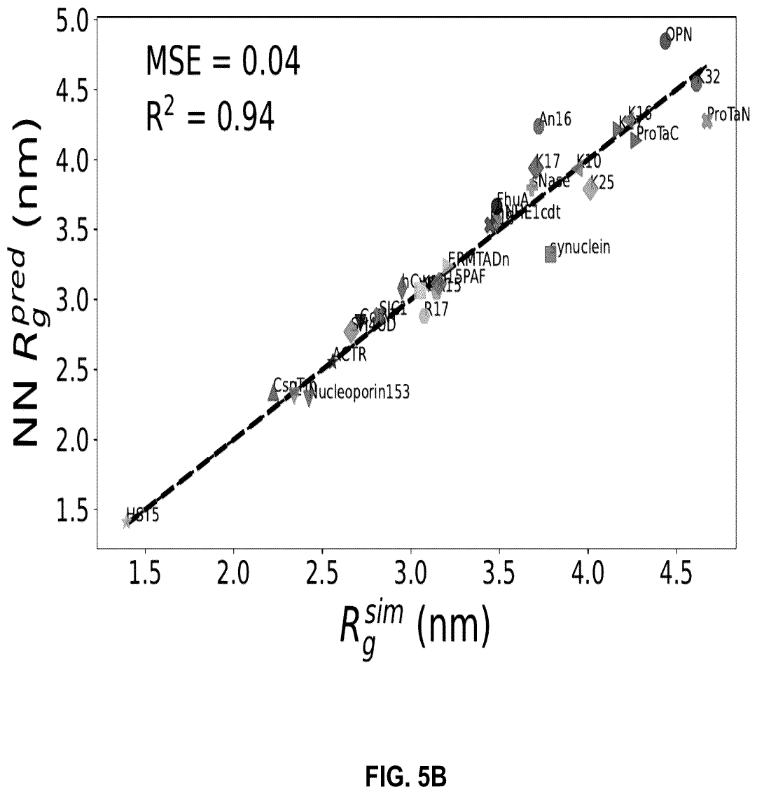

The trained neural network resulting from step 359 derives the [symbol] R_{g} [symbol] or [symbol] R_{N} [symbol] , or both, much faster than the computationally complex methods previously known and described above with respect to step 355. This speed provides many advantages for estimating the [symbol] R_{g} [symbol] and [symbol] R_{N} [symbol] for proteins not already in the library in various embodiments, such as mutations thereof. The accuracy of the trained network in an example embodiment is described below with reference to FIG. 5A and FIG. 5B.

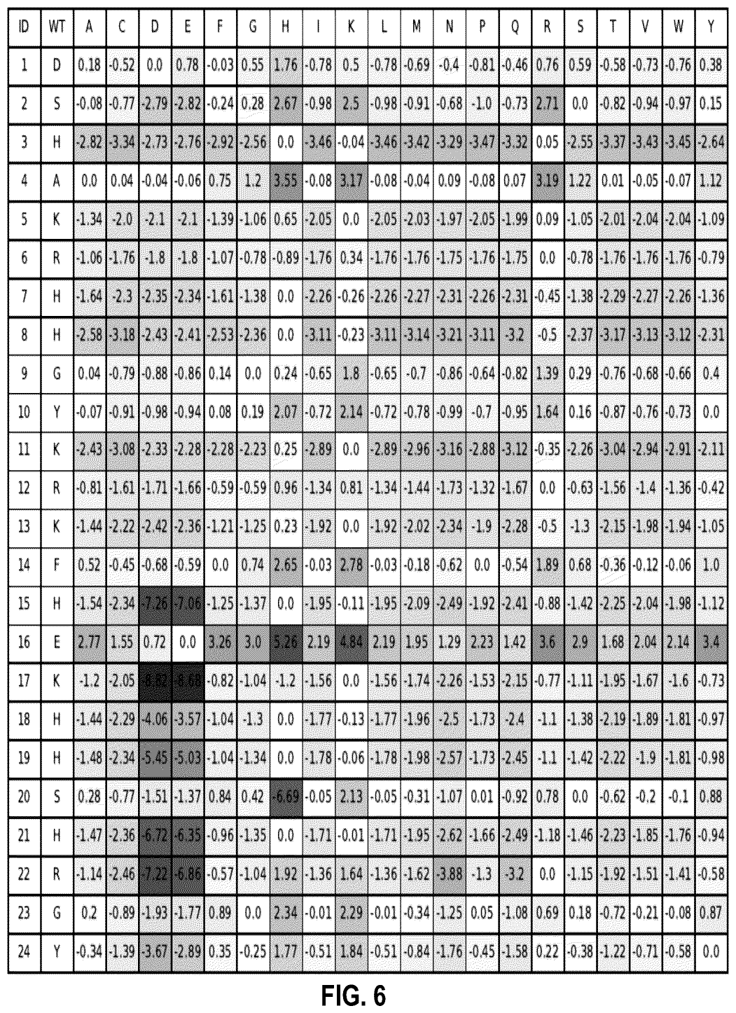

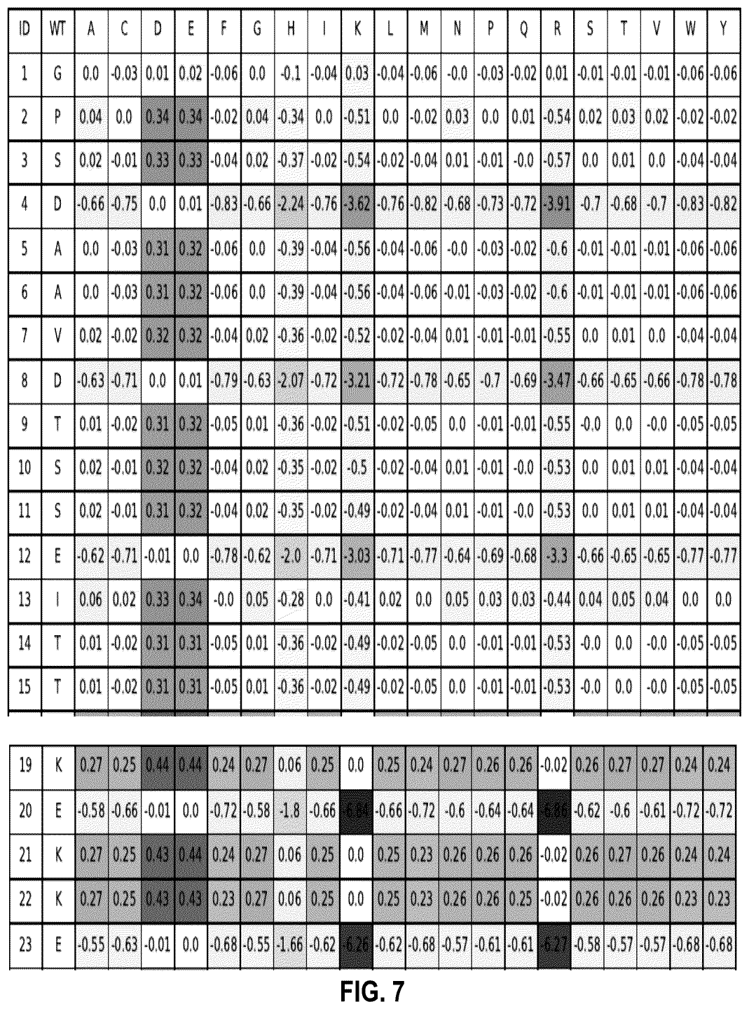

In one set of embodiments, the steps 361 through 375 are included to use the accurately trained neural network to efficiently and quickly determine the effect of millions of mutations for one or more important proteins involved in cell processes, regulation or disease. These results are depicted for one or more embodiments in FIG. 6 and FIG. 7 below.

In step 361, an amino acid sequence of an IDP or IDR of interest is selected, randomly or otherwise, with known (or tediously computed) gyration radius [symbol] R_{g} [symbol] or end to end distance [symbol] R_{N} [symbol] , or both. In step 363, a mutation is randomly introduced to the genes that produce the amino acid sequence of interest to produce a mutated sequence of amino acids. A vector of values for the multiple physical properties of the mutation are computed using the polymer physics-based computations, such as those described above for the 21 physics-based properties. In step 365, the vector of values for the multiple physical properties of the mutation are used as input to the trained neural network to output quick values for the gyration radius [symbol] R_{g} [symbol] or end-end distance [symbol] R_{N} [symbol] . or both.

In step 371, it is determined whether the change from the known [symbol] R_{g} [symbol] or [symbol] R_{N} [symbol] to the quick [symbol] R_{g} [symbol] or [symbol] R_{N} [symbol] is greater than some threshold of importance. The threshold can be set in any manner suitable for the purpose, such as a change that is greater than a certain percent of the original known value, e.g., greater than 30% or greater than 50% or greater than 75% or some other percentile. In other embodiments, the threshold is selected as a certain percentile of all the differences of all the mutations in a study. For example, one can start with a threshold of 20% and continue to refine the model by checking the distributions of the end-to-end distance and for the gyration radii, if they are very different compared to the wild type and continue to refine the threshold. Actual mutation data are used to gain confidence with the threshold value.

If it is determined in step 371 that the change (e.g., the difference in gyration radius) is not greater than the threshold, control passes to step 375, described below. If the change is greater than the threshold, control passes to step 373 to determine the detailed effect of the mutation. For example, by performing the complex and time consuming simulation described above with respect to step 355 on the mutated sequence. In some embodiments, step 373 includes wet laboratory experiments to induce the mutation and observe its effects. In some embodiments, step 373 includes a statistical or clinical study to search for the mutation as a factor separating healthy and diseased individuals in a population. Control then passes to step 375.

In step 375, it is determined whether there is another mutation to consider. If so, control passes back to step 361 and following steps, described above. If not, the process ends.

### NN Structure Details.

FIG. 4 is a block diagram that illustrates an example of a neural network structure, according to an embodiment. This structure was found to be effective in accurately predicting, within reasonable error bars in most cases, gyrations of mutated proteins, when compared to full scale modeling results. The neural network results were compared with simulation results for the IDPs which were not present in the training set. Neural network prediction mean-square-error (MSE) was found 0.04, hence achieving ˜95% accuracy.

The example neural network has an input layer 402 of 21 nodes, each node holding the value of one of the twenty-one physics-based properties derived from an amino acid sequence, such as an amino acid sequence of any length from the training set or the amino acid sequence of a randomly selected mutation of any length. In other embodiments fewer input nodes are used, such as five to ten input nodes for the five to ten most influential physics-based properties as determined statistically, such as by extra tree regressors or SHapley Additive explanations (SHAP) analysis. SHAP analysis is a method for interpreting machine learning model predictions by quantifying the contribution of individual input features to the overall output. It uses Shapley values, a concept from game theory, to explain how much each feature affects a single prediction. Essentially, SHAP analysis decomposes a model's prediction into contributions from each feature, allowing for a deeper understanding of how the model arrives at its conclusions. This analysis can identify a fewer number of input parameters that, in principle, is capable of making predictions with the same level of accuracy and, thus, avoid both overfitting and excessive computations Thus, explainable AI (SHAP) analysis can be used to estimate the lower number of physics inspired feature required to get a reasonable accuracy >90%. The mean SHAP value determines the average impact of one physics inspired parameter on the neural network output. For example, in one embodiment, the top 10 features are the most impactful features for accuracy of the neural network prediction.

The first hidden layer 421 represents many possible combinations of these 21 properties and thus has more than 21 nodes. It was found based on performance that the number of nodes in the first hidden layer is advantageously selected between two and four times the number of input nodes. For example, 32 nodes are used in the first hidden layer. second layer which is double the number of nodes present in the first hidden layer. One can choose a larger size, though not needed for our case and hyper-parameter tuning can also be used to determine the optimal number of nodes and number of hidden layers

The second hidden layer 422 starts converging on the limited number of output nodes. It was found, based on performance, that the number of nodes in the second hidden layer is advantageously selected to be 64. This is double the number of nodes present in the first hidden layer. One can choose a larger size, though not needed for our case and hyper-parameter tuning can also be used to determine the optimal number of nodes and number of hidden layers In other embodiments, more or fewer nodes of the same order of magnitude may be found to provide similar performance. The second hidden layer uses the ReLU activation function, described above, which allows only positive, switched but unsaturated values. This choice was made because ReLU activation function is known to preserve larger gradients and alleviates vanishing gradient problem and promotes faster convergence. The nodes are fully connected (dense) to the nodes in the previous layer, but not all are connected during each training instance. In neural networks, a dropout rate is the probability of a neuron being randomly deactivated during training. This helps prevent overfitting by forcing the network to learn more robust and generalizable representations. A commonly used range for dropout rates is 0.2 to 0.5. In the example embodiment, the dropout rate from the first hidden layer is 0.3.

The third hidden layer 423 has 32 fully connected (dense) nodes which is half the previous layer to allow combination of the more important features. The third hidden layer uses the tanh activation function, described above, which allows positive and negative but quickly switched and saturated values between −1 and 1. The tanh activation function is used to rescale and center the activations after a ReLU layer, helping maintain gradients in a reasonable range. This can prevent exploding activations in deep networks. In the example embodiment, the dropout rate from the second hidden layer during training is 0.2. The reduction from 64 nodes to 32 nodes represents a pooling factor of two to one. In other embodiments, more or fewer nodes of the same order of magnitude may be found to provide similar performance.

The fourth hidden layer 424 has 16 fully connected (dense) nodes, half the previous layer to again allow combination of the important features. The fourth hidden layer uses the ReLU activation function like the second hidden layer and for similar reasons. In the example embodiment, the dropout rate from the third hidden layer during training is 0.2. The reduction from 32 nodes to 16 nodes represents a pooling factor of two to one. In other embodiments, more or fewer nodes of the same order of magnitude may be found to provide similar performance.

The fifth hidden layer 425 has 8 fully connected (dense) nodes, again halving the number from the layer before for the same reasons. The fifth hidden layer uses the tanh activation function like the third hidden layer and for similar reasons. In the example embodiment, there is no dropout during training. The reduction from 16 nodes to 8 nodes represents a pooling factor of two to one. In other embodiments, more or fewer nodes of the same order of magnitude may be found to provide similar performance.

The sixth hidden layer 426 has 4 fully connected (dense) nodes. The sixth hidden layer uses the ReLU activation function like the fourth hidden layer, thus maintaining the pattern of alternating between the ReLU and tanh activation function. In the example embodiment, there is no dropout during training. The reduction from 8 nodes to 4 nodes represents a pooling factor of two to one. In other embodiments, more or fewer nodes of the same order of magnitude may be found to provide similar performance.

The output layer 404 has one connected (dense) node indicating the gyration radius or end-to-end distance, In another embodiment (not shown) the output layer 404 has two fully connected nodes indicating both. The output layer uses the linear activation function which exhibits neither switching nor saturation and allows both positive and negative values of any size.

Thus, the example neural network includes multiple fully connected (dense) hidden layers, with drop rates between 0.2 and 0.3, with pooling of at least a factor of two to one, with alternate hidden layers alternating between a tanh activation function and a RELU activation function, and the output layer using a linear activation function.

A CG simulation takes 5 hours per mutation sequence of 129 amino acids, while the NN takes less than 30 second for 19×129=2,451 mutants, which is less than 13 milliseconds (ms, 1 ms=10⁻³ seconds) per mutation. The time savings is a factor of almost one and half million (1.5M) to one.

### NN Accuracy.

FIG. 5A is a chart that illustrates an example of accuracy of the neural network in predicting gyrations of amino acid sequences, according to an example embodiment. In this chart, 31 different IDPs from a validation set are listed, one per line, in the first column. These IDPs were not used during training of the neural network. The second column lists the length of the amino acid sequence in number of amino acids in the protein for each IDP. The next column gives the gyration radius R_{g} in nanometers (nm, 1 nm=10⁻⁹ meters) as computed using the full, complex, multi-day CG simulation, labeled R_{g}^{HPS2} for each IDP. HPS means Hydropathy Scale—HPS2 is a hydropathy scale due to Tesei et al. and is explained in Seth and Bhattacharya “Fine structures of intrinsically disordered proteins”, JCP, incorporated by reference as if fully set forth herein. The next column gives the gyration radius R_{g} as output quickly by the neural network of FIG. 4, labeled R_{g}^{NN} for each IDP. The final column, labeled ΔR_{g}, gives the difference in percent, 100 (R_{g}^{HPS2}−R_{g}^{NN})/R_{g}^{HPS2}, for each IDP. As can be seen, the average agreement is within 4%, varying between-14% and +13%.

As demonstrated in FIG. 5A and FIG. 5B, neural network predictions give about 96% accurate agreement with the simulation data. ProTaN is one of the exceptions. This is believed to be due to the fact that ProTaN has a negative charge patch of Glutamic acid and Aspartic acid repeat sequence, yielding a higher gyration radius.

FIG. 5B is a plot that illustrates an example of accuracy of the neural network in predicting gyrations of mutations, according to an example embodiment. The horizontal axis indicates the gyration radius R_{g} in nanometers (nm) as computed using the full, complex, multi-day CG simulation, here re-labeled R_{g}^{sim} for each IDP. The vertical axis indicates the gyration radius R_{g} in nanometers (nm) as computed instantly using the neural network of FIG. 4, here re-labeled R_{g}^{pred} for each IDP. Perfect agreement is along the dashed line. As can be seen, the neural network predicted gyration radii are useful approximations, at a tiny fraction of the time, for the more reliable but days-slow simulated gyration radii.

### Example Use of Predictive Model

The values of gyration radii, or end-to-end distances, or both, output by the fast neural network can then be used to quickly identify mutations with large enough differences from the wild type to be of concern. These mutations of concern can then be simulated more carefully using the days-slow conventional methods. FIG. 6 and FIG. 7 are charts that illustrate further examples of subsets of mutations with significant differences in gyrations from corresponding unmutated IDPs, according to two further example embodiments.

The chart in FIG. 6 lists the gyration radius differences for single AA mutations of a wild type IDP with a 24 amino acid long sequence. The amino acid sequence of the wild type is given by the amino acid letters listed in the column labeled WT. The gyration radius of the wild type IDP is known or pre-computed using the gold standard simulation. The next 20 columns are labeled with an amino acid letter of an amino acid that is substituted for each one of the 24 amino acids in the wild type. A single amino acid in a protein is also called a residue. Each cell in the chart gives the gyration radius difference, in percentage (%), between the known gyration radius for the wild type and the gyration radius of the mutation estimated by the trained neural network. Thus, for each of 24 wild type residues in the WT sequence, there are 20 possible residue substitutions leading to 24×20=480 cells. When the substituted residue is the same as the wild type residue at a location along the sequence, the difference in gyration radius should be small, on the order of the error in the neural network prediction, i.e., having an absolute value error less than 0.1 nm. Such negligible errors can be seen in the chart wherever a column and row have the same letter. Thus, there are 480 cells, but only 480−24=456 mutations. Because the chart is based on gyration radii computed by the quick neural network, these are called quick radii, and the differences are quick differences.

In the chart of FIG. 6, cells are darkened according to the size of the quick difference, so that the darkest cells have the biggest quick gyration radius difference. The darkest cells are the mutations of concern, and in some embodiments are then subjected to further simulation or modeling to provide confidence in their status as significant mutations, even pathogenic mutations. As can be seen, the darkest cells number about 10 to 20 out of a set of 456 mutations (2% to 4%), a reduction by 96% to 98%, almost two orders of magnitude, in number of mutations subjected to days-long simulations and further study. The savings in time is substantial, about 2,000 to 4,000 hours, which is one to two person years.

In FIG. 6, the greatest effect is observed for some substitutions at positions 15, 16 and 17 of the wild type. A pattern that emerges is that substituting a neutral residue for a charged residue has an increased effect.

The chart in FIG. 7 lists the gyration radius differences for single AA mutations of a wild type histidine IDP, ProTa-N with a 112 amino acid long sequence, of which only positions 1 to 15 and 19-23 are shown for brevity. Otherwise, the chart rows and columns and cells are defined the same as in FIG. 6. Substitutions of residues Dand E have the greatest effect. These two letters represent charged residues aspartate (Asp) and glutamate (Glu), respectively. One can conclude that mutations induced by insertion of charged residues Asp or Glu can have a prominent effect, specifically to histidine.

The percentage savings is even greater with longer IDPs. For example, similar determinations of quick gyration radii differences were conducted for IDPs CspTm, Prota-C, and P53 with sequence lengths 67, 129, and 93, respectively. The neural network has yielded a 10,000-fold reduction in the search space for potentially harmful mutations.

### System Hardware

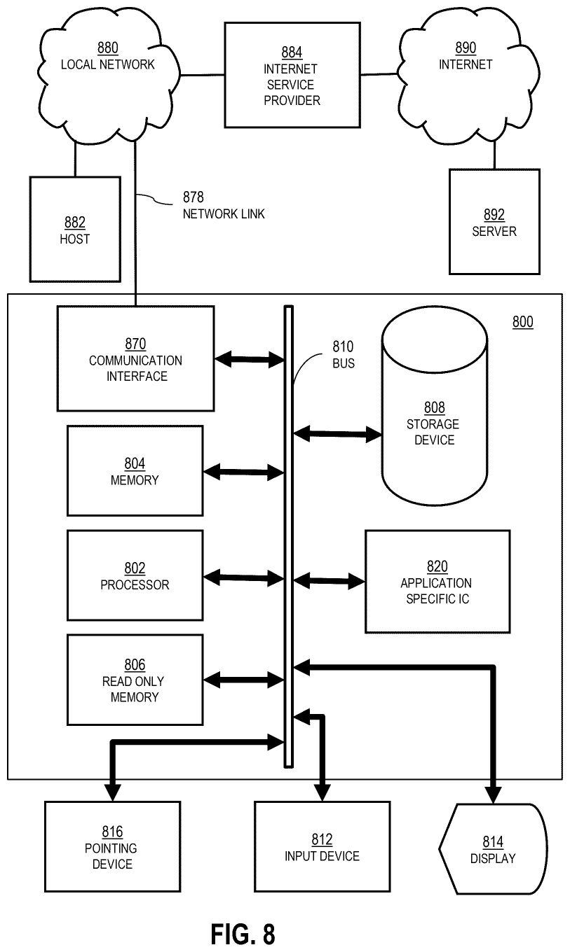

FIG. 8 is a block diagram that illustrates a computer system 800 upon which an embodiment of the invention may be implemented. Computer system 800 includes a communication mechanism such as a bus 810 for passing information between other internal and external components of the computer system 800. Information is represented as physical signals of a measurable phenomenon, typically electric voltages, but including, in other embodiments, such phenomena as magnetic, electromagnetic, pressure, chemical, molecular atomic and quantum interactions. For example, north and south magnetic fields, or a zero and non-zero electric voltage, represent two states (0, 1) of a binary digit (bit).). Other phenomena can represent digits of a higher base. A superposition of multiple simultaneous quantum states before measurement represents a quantum bit (qubit). A sequence of one or more digits constitutes digital data that is used to represent a number or code for a character. In some embodiments, information called analog data is represented by a near continuum of measurable values within a particular range. Computer system 800, or a portion thereof, constitutes a means for performing one or more steps of one or more methods described herein.

A sequence of binary digits constitutes digital data that is used to represent a number or code for a character. A bus 810 includes many parallel conductors of information so that information is transferred quickly among devices coupled to the bus 810. One or more processors 802 for processing information are coupled with the bus 810. A processor 802 performs a set of operations on information. The set of operations include bringing information in from the bus 810 and placing information on the bus 810. The set of operations also typically include comparing two or more units of information, shifting positions of units of information, and combining two or more units of information, such as by addition or multiplication. A sequence of operations to be executed by the processor 802 constitutes computer instructions.

Computer system 800 also includes a memory 804 coupled to bus 810. The memory 804, such as a random access memory (RAM) or other dynamic storage device, stores information including computer instructions. Dynamic memory allows information stored therein to be changed by the computer system 800. RAM allows a unit of information stored at a location called a memory address to be stored and retrieved independently of information at neighboring addresses. The memory 804 is also used by the processor 802 to store temporary values during execution of computer instructions. The computer system 800 also includes a read only memory (ROM) 806 or other static storage device coupled to the bus 810 for storing static information, including instructions, that is not changed by the computer system 800. Also coupled to bus 810 is a non-volatile (persistent) storage device 808, such as a magnetic disk or optical disk, for storing information, including instructions, that persists even when the computer system 800 is turned off or otherwise loses power.

Information, including instructions, is provided to the bus 810 for use by the processor from an external input device 812, such as a keyboard containing alphanumeric keys operated by a human user, or a sensor. A sensor detects conditions in its vicinity and transforms those detections into signals compatible with the signals used to represent information in computer system 800. Other external devices coupled to bus 810, used primarily for interacting with humans, include a display device 814, such as a cathode ray tube (CRT) or a liquid crystal display (LCD), for presenting images, and a pointing device 816, such as a mouse or a trackball or cursor direction keys, for controlling a position of a small cursor image presented on the display 814 and issuing commands associated with graphical elements presented on the display 814.

In the illustrated embodiment, special purpose hardware, such as an application specific integrated circuit (IC) 820, is coupled to bus 810. The special purpose hardware is configured to perform operations not performed by processor 802 quickly enough for special purposes. Examples of application specific ICs include graphics accelerator cards for generating images for display 814, cryptographic boards for encrypting and decrypting messages sent over a network, speech recognition, and interfaces to special external devices, such as robotic arms and medical scanning equipment that repeatedly perform some complex sequence of operations that are more efficiently implemented in hardware.

Computer system 800 also includes one or more instances of a communications interface 870 coupled to bus 810. Communication interface 870 provides a two-way communication coupling to a variety of external devices that operate with their own processors, such as printers, scanners and external disks. In general the coupling is with a network link 878 that is connected to a local network 880 to which a variety of external devices with their own processors are connected. For example, communication interface 870 may be a parallel port or a serial port or a universal serial bus (USB) port on a personal computer. In some embodiments, communications interface 870 is an integrated services digital network (ISDN) card or a digital subscriber line (DSL) card or a telephone modem that provides an information communication connection to a corresponding type of telephone line. In some embodiments, a communication interface 870 is a cable modem that converts signals on bus 810 into signals for a communication connection over a coaxial cable or into optical signals for a communication connection over a fiber optic cable. As another example, communications interface 870 may be a local area network (LAN) card to provide a data communication connection to a compatible LAN, such as Ethernet. Wireless links may also be implemented. Carrier waves, such as acoustic waves and electromagnetic waves, including radio, optical and infrared waves travel through space without wires or cables. Signals include man-made variations in amplitude, frequency, phase, polarization or other physical properties of carrier waves. For wireless links, the communications interface 870 sends and receives electrical, acoustic or electromagnetic signals, including infrared and optical signals that carry information streams, such as digital data.

The term computer-readable medium is used herein to refer to any medium that participates in providing information to processor 802, including instructions for execution. Such a medium may take many forms, including, but not limited to, non-volatile media, volatile media and transmission media. Non-volatile media include, for example, optical or magnetic disks, such as storage device 808. Volatile media include, for example, dynamic memory 804. Transmission media include, for example, coaxial cables, copper wire, fiber optic cables, and waves that travel through space without wires or cables, such as acoustic waves and electromagnetic waves, including radio, optical and infrared waves. The term computer-readable storage medium is used herein to refer to any medium that participates in providing information to processor 802, except for transmission media.

Common forms of computer-readable media include, for example, a floppy disk, a flexible disk, a hard disk, a magnetic tape, or any other magnetic medium, a compact disk ROM (CD-ROM), a digital video disk (DVD) or any other optical medium, punch cards, paper tape, or any other physical medium with patterns of holes, a RAM, a programmable ROM (PROM), an erasable PROM (EPROM), a FLASH-EPROM, or any other memory chip or cartridge, a carrier wave, or any other medium from which a computer can read. The term non-transitory computer-readable storage medium is used herein to refer to any medium that participates in providing information to processor 802, except for carrier waves and other signals.

Logic encoded in one or more tangible media includes one or both of processor instructions on a computer-readable storage media and special purpose hardware, such as ASIC 820.

Network link 878 typically provides information communication through one or more networks to other devices that use or process the information. For example, network link 878 may provide a connection through local network 880 to a host computer 882 or to equipment 884 operated by an Internet Service Provider (ISP). ISP equipment 884 in turn provides data communication services through the public, world-wide packet-switching communication network of networks now commonly referred to as the Internet 890. A computer called a server 892 connected to the Internet provides a service in response to information received over the Internet. For example, server 892 provides information representing video data for presentation at display 814.

The invention is related to the use of computer system 800 for implementing the techniques described herein. According to one embodiment of the invention, those techniques are performed by computer system 800 in response to processor 802 executing one or more sequences of one or more instructions contained in memory 804. Such instructions, also called software and program code, may be read into memory 804 from another computer-readable medium such as storage device 808. Execution of the sequences of instructions contained in memory 804 causes processor 802 to perform the method steps described herein. In alternative embodiments, hardware, such as application specific integrated circuit 820, may be used in place of or in combination with software to implement the invention. Thus, embodiments of the invention are not limited to any specific combination of hardware and software.

The signals transmitted over network link 878 and other networks through communications interface 870, carry information to and from computer system 800. Computer system 800 can send and receive information, including program code, through the networks 880, 890 among others, through network link 878 and communications interface 870. In an example using the Internet 890, a server 892 transmits program code for a particular application, requested by a message sent from computer 800, through Internet 890, ISP equipment 884, local network 880 and communications interface 870. The received code may be executed by processor 802 as it is received, or may be stored in storage device 808 or other non-volatile storage for later execution, or both. In this manner, computer system 800 may obtain application program code in the form of a signal on a carrier wave.

Various forms of computer readable media may be involved in carrying one or more sequence of instructions or data or both to processor 802 for execution. For example, instructions and data may initially be carried on a magnetic disk of a remote computer such as host 882. The remote computer loads the instructions and data into its dynamic memory and sends the instructions and data over a telephone line using a modem. A modem local to the computer system 800 receives the instructions and data on a telephone line and uses an infra-red transmitter to convert the instructions and data to a signal on an infra-red a carrier wave serving as the network link 878. An infrared detector serving as communications interface 870 receives the instructions and data carried in the infrared signal and places information representing the instructions and data onto bus 810. Bus 810 carries the information to memory 804 from which processor 802 retrieves and executes the instructions using some of the data sent with the instructions. The instructions and data received in memory 804 may optionally be stored on storage device 808, either before or after execution by the processor 802.

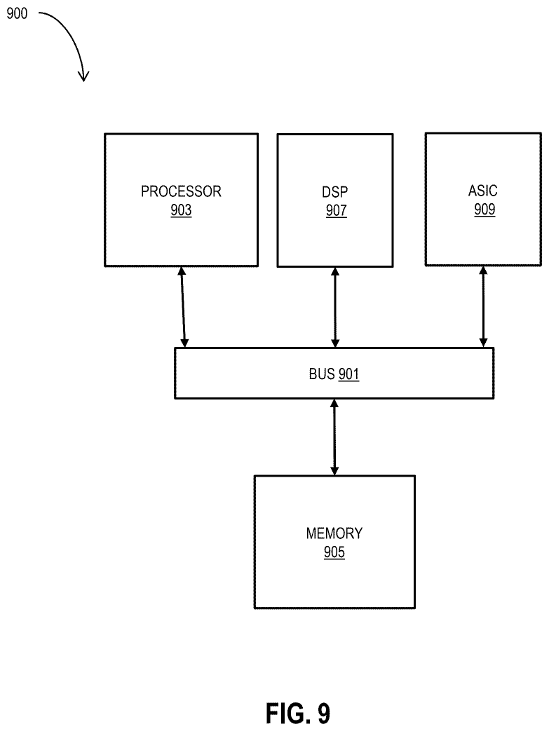

FIG. 9 illustrates a chip set 900 upon which an embodiment of the invention may be implemented. Chip set 900 is programmed to perform one or more steps of a method described herein and includes, for instance, the processor and memory components described with respect to FIG. 8 incorporated in one or more physical packages (e.g., chips). By way of example, a physical package includes an arrangement of one or more materials, components, and/or wires on a structural assembly (e.g., a baseboard) to provide one or more characteristics such as physical strength, conservation of size, and/or limitation of electrical interaction. It is contemplated that in certain embodiments the chip set can be implemented in a single chip. Chip set 900, or a portion thereof, constitutes a means for performing one or more steps of a method described herein.

In one embodiment, the chip set 900 includes a communication mechanism such as a bus 901 for passing information among the components of the chip set 900. A processor 903 has connectivity to the bus 901 to execute instructions and process information stored in, for example, a memory 905. The processor 903 may include one or more processing cores with each core configured to perform independently. A multi-core processor enables multiprocessing within a single physical package. Examples of a multi-core processor include two, four, eight, or greater numbers of processing cores. Alternatively or in addition, the processor 903 may include one or more microprocessors configured in tandem via the bus 901 to enable independent execution of instructions, pipelining, and multithreading. The processor 903 may also be accompanied with one or more specialized components to perform certain processing functions and tasks such as one or more digital signal processors (DSP) 907, or one or more application-specific integrated circuits (ASIC) 909. A DSP 907 typically is configured to process real-world signals (e.g., sound) in real time independently of the processor 903. Similarly, an ASIC 909 can be configured to performed specialized functions not easily performed by a general purposed processor. Other specialized components to aid in performing the inventive functions described herein include one or more field programmable gate arrays (FPGA) (not shown), one or more controllers (not shown), or one or more other special-purpose computer chips.

The processor 903 and accompanying components have connectivity to the memory 905 via the bus 901. The memory 905 includes both dynamic memory (e.g., RAM, magnetic disk, writable optical disk, etc.) and static memory (e.g., ROM, CD-ROM, etc.) for storing executable instructions that when executed perform one or more steps of a method described herein. The memory 905 also stores the data associated with or generated by the execution of one or more steps of the methods described herein.

### Kits and Assays

The phrase “high throughput screen” or a “high throughput screening method” as used herein defines a process in which large numbers of test agents, e.g., compounds, are tested rapidly and in parallel for the ability to reduce aberrant folding of an intrinsically disordered protein. In certain embodiments, “large numbers of agents, e.g., compounds” may be, for example, more than 100 or more than 300 or more than 500 or more than 1,000 compounds. Preferably, the process is an automated process.

“Test agent” or “test compound” includes any chemical or biological factor that is used in the methods of the invention, whether new (i.e., a “new chemical entity” or NCE) or known (e.g., a small molecule drug lead or small molecule already-approved drug), that is administered to or contacted with one or more cells gene products (e.g. expressed proteins) for the purpose of screening it for activity toward the protection of the gene product from disordered or aberrant conformation changes or folding.

### Drug Screening Assays

As used herein, “high throughput screening” refers to a method that allows a researcher to quickly conduct chemical, genetic or pharmacological tests, the results of which provide starting points for drug design and for understanding the interaction or role of a particular biochemical process in biology. High-throughput screening methods known in the art are used to screen thousands of new or known test agents to identify potential therapeutic drugs in vitro for their ability to protect against aberrant folding of mutated proteins, which greatly accelerates drug development and renders it safer and cheaper than having to test all agents in biological assays. In certain embodiments, the high-throughput screening is accomplished in vitro. In an embodiment, the method is used to screen a library of compounds. In this context, the library of compounds may be composed of a plurality of chemical substances that may be assembled of multiple sources as is described below.

In the context of the present invention the term “screen” relates to a method in which a standardized molecular assay or a composition of several molecular assays is applied to a plurality of compounds to determine their properties of interest such as the particular ability to significantly reduce aberrant folding or function of a gene product.

A screen may be carried out in solution, e.g., in flasks, reaction tubes, cuvettes, microtiter plates and the like, for example in a microarray format. The screen may preferably be carried out with little compound consumption and/or small volumes. High throughput robotic screening on extremely few cells, sometimes even on a single cell, is preferred, therefore the use of a microtiter format is a typical implementation. On such a microtiter plate, small amounts such as only a few microliters may be sufficient for the screen.

In an embodiment, a high-throughput screening of the test agents identifies and selects those test agents that significantly reduce aberrant conformational changes or dysfunction of intrinsically disordered proteins.

In a preferred embodiment, the high-throughput screening is carried out in an automated format, particularly in a high-throughput format. In the context of the present invention, the term “automated format” refers to a method that is fully or partly controlled and/or carried out by one or more technical devices, preferably pipetting robots. In this context, the term “high-throughput format” relates to a screen/assay system for the rapid testing of a plurality of compounds within in a short time, thus, the screening/assaying time per tested compound is minimized. The initial screen of test agents is preferably carried out in multi-well plates in which an isolated IDP is located, more preferably in E-well plates, 12-well plates, 24-well plates, 96-well plates or 384-well plates, even more preferably in 96-well plates or 384-well plates.

### Library

Test agents for use in screening encompass numerous chemical classes, though typically they are organic molecules, preferably small organic compounds having a molecular weight of more than 100 and less than about 2,500 daltons (Da), preferably less than about 500 Da. Some test agents comprise functional groups that permit them to structurally interaction with proteins, particularly hydrogen bonding, and typically include at least an amine, carbonyl, hydroxyl or carboxyl group, preferably at least two of the functional chemical groups. Such agents often comprise cyclical carbon or heterocyclic structures and/or aromatic or polyaromatic structures substituted with one or more of the above functional groups. Test agents are also found among biomolecules including peptides, saccharides, fatty acids, steroids, purines, pyrimidines, derivatives, structural analogs or combinations thereof. Libraries of high-purity small organic ligands and peptides that have well-documented pharmacological activities are available from numerous sources. One example is an NCI diversity set which contains 1,886 drug-like compounds (small, intermediate hydrophobicity). Another is an Institute of Chemistry and Cell Biology (ICCB; maintained by Harvard Medical School) set of known bioactives (467 compounds) which includes many extended, flexible compounds. Some other examples of the ICCB libraries are: Chem Bridge DiverSet E (16,320 compounds); Bionet 1 (4,800 compounds); CEREP (4,800 compounds); Maybridge 1 (8,800 compounds); Maybridge 2 (704 compounds); Maybridge HitFinder (14,379 compounds); Peakdale 1 (2,816 compounds); Peakdale 2 (352 compounds); GhemDiv Combilab and International (28,864 compounds); Mixed Commercial Plate 1 (352 compounds); Mixed Commercial Plate 2 (320 compounds); Mixed Commercial Plate 3 (251 compounds); Mixed Commercial Plate 4 (331 compounds); ChemBridge Microformat (50,000 compounds); Commercial Diversity Set1 (5,056 compounds). Other NCI Collections are: Structural Diversity Set, version 2 (1,900 compounds); Mechanistic Diversity Set (879 compounds); Open Collection 1 (90,000 compounds); Open Collection 2 (10,240 compounds); Known Bioactives Collections: NINDS Custom Collection (1,040 compounds); ICCB Bioactives 1 (489 compounds); SpecPlus Collection (960 compounds); ICCB Discretes Collections. The following ICCB compounds were collected individually from chemists at the ICCB, Harvard, and other collaborating institutions: ICCB1 (190 compounds); ICCB2 (352 compounds); ICCB3 (352 compounds); ICCB4 (352 compounds). Natural Product Extracts: NCI Marine Extracts (352 wells); Organic fractions—NC! Plant and Fungal Extracts (1,408 wells); Philippines Plant Extracts 1 (200 wells); ICCB-ICG Diversity Oriented Synthesis (DOS) Collections; DDS1 (DOS Diversity Set) (9600 wells). Compound libraries are also available from commercial suppliers, such as ActiMol, Albany Molecular, Bachem, Sigma-Aldrich, TimTec, and others.

The library may be fully randomized, with no sequence preferences or constants at any position. The library may be biased. That is, some positions within the sequence are either held constant, or are selected from a limited number of possibilities. For example, the nucleotides or amino acid residues are randomized within a defined class, for example, of hydrophobic amino acids, hydrophilic residues, sterically biased (either small or large) residues, towards the creation of cysteines, for cross-linking, prolines for SH-3 domains, serines, threonines, tyrosines or histidines for phosphorylation sites, etc., or to purines, etc.

The phrase “small organic” or “small inorganic” molecule includes any chemical or other moiety, other than polysaccharides, polypeptides, and nucleic acids, that can act to affect biological processes. Small molecules can include any number of therapeutic agents presently known and used, or can be synthesized in a library of such molecules for the purpose of screening for biological function(s). Small molecules are distinguished from macromolecules by size. The small molecules of this invention usually have a molecular weight less than about 5,000 daltons (Da), preferably less than about 2,500 Da, more preferably less than 1,000 Da, most preferably less than about 500 Da.

As used herein, the term “organic compound” refers to any carbon-based compound other than macromolecules such as nucleic acids and polypeptides. In addition to carbon, organic compounds may contain calcium, chlorine, fluorine, copper, hydrogen, iron, potassium, nitrogen, oxygen, sulfur and other elements. An organic compound may be in an aromatic or aliphatic form. Non-limiting examples of organic compounds include acetones, alcohols, anilines, carbohydrates, mono-saccharides, di-saccharides, amino acids, nucleosides, nucleotides, lipids, retinoids, steroids, proteoglycans, ketones, aldehydes, saturated, unsaturated and polyunsaturated fats, oils and waxes, alkenes, esters, ethers, thiols, sulfides, cyclic compounds, heterocyclic compounds, imidizoles, and phenols. An organic compound as used herein also includes nitrated organic compounds and halogenated (e.g., chlorinated) organic compounds. Collections of small molecules, and small molecules identified according to the invention, are characterized by techniques such as accelerator mass spectrometry {A S; see Turteltaub et al., Curr Pharm Des 2000 6:991-1007, Bioanalytical applications of accelerator mass spectrometry for pharmaceutical research; and Enjalbal et al., Mass Spectrom Rev 2000 19:139-61, Mass spectrometry in combinatorial chemistry.)

Preferred small molecules are relatively easier and less expensively manufactured, formulated or otherwise prepared. Preferred small molecules are stable under a variety of storage conditions. Preferred small molecules may be placed in tight association with macromolecules to form molecules that are biologically active and that have improved pharmaceutical properties. Improved pharmaceutical properties include changes in circulation time, distribution, metabolism, modification, excretion, secretion, elimination, and stability that are favorable to the desired biological activity. Improved pharmaceutical properties include changes in the toxicological and efficacy characteristics of the chemical entity.

“Compound” and “agent” are used interchangeably herein to describe any composition of matter including a chemical entity or a biological factor that is administered, approved or under testing as potential therapeutic agent or is a known therapeutic agent. Thus the term encompasses chemical entities and biological factors as defined, infra.

Any library of chemical compounds/agents available or generated by a person skilled in the art can be applied to methods of the invention to screen the provided compounds agents from the library for their ability to significantly reduce aberrant conformational changes and/or activity of IDPs. Preferably, one or more compounds/agents are identified from a group of compounds, preferably from a compound library. As used herein the term “identifying a compound” may be understood as interchangeable with “detection of a compound” or “finding a compound.” The term “identifying” herein may be understood as a relative term meaning that the test compound/agent has the desired biological activity of reducing aberrant conformational changes and/or activity of IDPs. Test agents that achieve such reduction are potential therapeutic agents that warrant further in vivo testing.

### Methods of Therapy and Medical Uses

The invention concerns methods of treatment of a retinal disease and vectors for use in such methods. The method may be a method of gene therapy. The term “gene therapy” means the therapeutic delivery of nucleic acid polymers into a patient's cells. In some cases of gene therapy, copies of one or more genes that encode a protein that is therapeutic to a subject are introduced to cells of the subject. Such a gene for introduction to the cells may be referred to herein as a transgene. In some cases the gene therapy introduces to the cells one or more genes that are normally expressed in a healthy individual but that are missing or defective in the subject. In other cases of gene therapy a nucleic acid polymer may be introduced to knock down expression of a gene in the subject, that is to reduce or inhibit expression of a gene product. According to the present invention the gene therapy comprises administration of a vector that comprises a mirtron for knocking down expression of a gene in a subject and a transgene for expressing a gene in the subject. The gene that is targeted for knock down by the mirtron may be referred to herein as an endogenous gene or target gene. The transgene may in some cases be referred to as an exogenous gene.

The disease that is treated may be any disease, condition or disorder that is caused by or exacerbated by the expression or over-expression of a gene in a subject. In some cases the disease may be caused by a mutation in the gene compared to a wild-type healthy gene. The disease may be dominant genetic (or autosomal dominant) in which a mutation in one of the two copies of the gene in a subject can be sufficient for the subject to be affected by the disease or for the disease to be exacerbated. In other cases the disease may be recessive genetic (or autosomal recessive), in which a subject must have mutations in both copies of the gene to be affected or for the disease to be exacerbated. In some cases the disease is caused by or primarily caused or triggered by mutation in a single gene. In other cases the present invention may be used to treat complex genetic diseases, in which multiple genes may be involved, possibly in combination with lifestyle or environmental contributory factors.

The subject may be a human or a non-human animal. Non-human animals include, but are not limited to, rodents (including mice and rats), and other common laboratory, domestic and agricultural animals, including rabbits, dogs, cats, horses, cows, sheep, goats, pigs, chickens, amphibians, reptiles etc.

As used herein, the term “amount” refers to “an amount effective” or “an effective amount” of a composition, polynucleotide, or viral vector contemplated herein sufficient to achieve a beneficial or desired prophylactic or therapeutic result, including clinical results.

As used herein, the term “amount” refers to “an amount effective” or “an effective amount” of a composition, polynucleotide, or viral vector contemplated herein sufficient to achieve a beneficial or desired prophylactic or therapeutic result, including clinical results.

A “prophylactically effective amount” refers to an amount of a composition, polynucleotide, or viral vector contemplated herein sufficient to achieve the desired prophylactic result. Typically but not necessarily, since a prophylactic dose is used in subjects prior to or at an earlier stage of disease, the prophylactically effective amount is less than the therapeutically effective amount.

A “therapeutically effective amount” of a virus may vary according to factors such as the disease state, age, sex, and weight of the individual, and the ability of the stem and progenitor cells to elicit a desired response in the individual. A therapeutically effective amount is also one in which any toxic or detrimental effects of a composition, polynucleotide, or viral vector contemplated herein are outweighed by the therapeutically beneficial effects. The term “therapeutically effective amount” includes an amount that is effective to “treat” a subject (e.g., a patient). In one example, a therapeutically effective amount is a sufficient amount of a gene therapy composition sufficient to correct or ameliorate the effects of an aberrant conformational or function of a gene product, such as an IDP.

As used herein “treatment” or “treating,” includes any beneficial or desirable effect associated with a reduction in one or more symptoms or other effects of a disease or condition associated with an aberrant conformational change or function of a gene product. “Treatment” does not necessarily indicate complete eradication or cure of the disease or condition, or associated symptoms thereof.

As used herein, “prevent,” and similar words such as “prevented,” “preventing” etc., indicate an approach for preventing, inhibiting, or reducing the likelihood of the occurrence or recurrence of one or more symptoms or other effects of a disease or condition associated with an aberrant conformational change or function of a gene product.

As used herein, “management” or “controlling” one or more symptoms or effects of a disease or condition.

### CRISPR

In another embodiment, an IDP detected or determined by the methods herein is corrected ex vivo or in vivo using a CRISPR-Cas system. Once the sequence of a critical mutation in a nucleic acid is correlated with a gene product leading to an IDP, constructs can be created that target and correct the mutation to that observed in a healthy individual using a CRISPR-Cas system. Multiple class 1 CRISPR-Cas systems, which include the type I and type Ill systems, have been identified and functionally characterized in detail, revealing the complex architecture and dynamics of the effector complexes (Brouns et al., 2008, Marraffini and Sontheimer, 2008, Hale et al., 2009, Sinkunas et al., 2013, Jackson et al., 2014, Mulepati et al., 2014). In addition, several class 2-type II CRISPR-Cas systems that employ homologous RNA-guided endonucleases of the Cas9 family as effectors have also been identified and experimentally characterized (Barrangou et al., 2007, Garneau et al., 2010, Deltcheva et al., 2011, Sapranauskas et al., 2011, Jinek et al., 2012, Gasiunas et al., 2012). A second, putative class 2-type V CRISPR-Cas system has been recently identified in several bacterial genomes. The putative type V CRISPR-Cas systems contain a large, ˜1,300 amino acid protein called Cpf1 (CRISPR from Prevotella and Francisella 1).

The CRISPR/Cas nuclease system can be used to introduce a double-strand break in a target polynucleotide sequence, which may be repaired by non-homologous end joining (NHEJ) in the absence of a polynucleotide template, e.g., a DNA template for altering at least one site in a genome, or by homology directed repair (HDR), i.e., homologous recombination, in the presence of a polynucleotide repair template. Cas9 and Cpf1 nucleases can also be engineered as nickases, which generate single-stranded DNA breaks that can be repaired using the cell's base-excision-repair (BER) machinery or homologous recombination in the presence of a repair template. NHEJ is an error-prone process that frequently results in the formation of small insertions and deletions that disrupt gene function. Homologous recombination requires homologous DNA as a template for repair and can be leveraged to create a limitless variety of modifications specified by the introduction of donor DNA containing the desired sequence flanked on either side by sequences bearing homology to the target.

In various embodiments, vectors contemplated herein contain polynucleotides to be expressed that are flanked by one or more crRNA or sgRNA target sites to transiently regulate the expression of the polynucleotide.

In one embodiment, wherein a crRNA or sgRNA is directed against a polynucleotide sequence encoding a polypeptide, NHEJ of the ends of the cleaved genomic sequence may result in a normal polypeptide, a loss-of- or gain-of-function polypeptide, or knock-out of a functional polypeptide.

In another embodiment, wherein a crRNA or sgRNA is directed against a polynucleotide sequence encoding a cis-acting sequence that regulates mRNA expression of a polynucleotide sequence encoding a polypeptide, NHEJ of the genomic sequence may result increased expression, decreased expression, or complete loss of expression of the mRNA and polypeptide.

In another embodiment, wherein a polynucleotide template for repair of the cleaved genomic sequence is provided, the genomic locus is repaired with the sequence of the template by homologous recombination. In one embodiment, the repair template comprises a polynucleotide sequence that is different from a targeted genomic sequence. In one embodiment, the repair template comprises one or more polynucleotides that restores function of the targeted genomic sequence or restores the natural polynucleotide sequence encoding a wild type allele of a polypeptide. In another embodiment, the repair DNA template comprises one or more polynucleotides that reduces or eliminates function of the targeted genomic sequence or decreases the expression of the natural polynucleotide sequence encoding a wild type allele of a polypeptide and/or increasing the expression of a variant polypeptide. In another embodiment, the repair DNA template comprises one or more expression control sequences or transcription regulatory sequences that regulates the transcriptional activity of the locus.

As used herein, the term “guide RNA” refers to a “crRNA” and/or an “sgRNA.”

As used herein, the term “crRNA” refers to an RNA comprising a region of partial or total complementarity referred to herein as a “spacer motif” to a target polynucleotide sequence referred to herein as a protospacer motif. In one embodiment, a protospacer motif is a 20 nucleotide target sequence. In particular embodiments, the protospacer motif is 18, 19, 20, 21, 22, 23, 24, 25, 26, 27, 28, 29, 30, or more nucleotides. Without wishing to be bound by any particular theory, it is contemplated that protospacer target sequences of various lengths will be recognized by different bacterial species.

In one embodiment, the region of complementarity comprises a polynucleotide sequence that is at least 80%, 81%, 82%, 83%, 84%, 85%, 86%, 87%, 88%, 89%, 90%, 91%, 92%, 93%, 94%, 95%, 96%, 97%, 98%, 99% or 100% identical to the protospacer sequence. In a related embodiment, at least 10, 11, 12, 13, 14, 15, 16, 17, 18, 19, or 20 or more polynucleotides in the region of complementarity are identical to the protospacer motif. In a preferred embodiment, at least 10 of 3′ most sequence in the protospacer motif is complementary to the crRNA sequence.

As used herein, the term “tracrRNA” refers to a trans-activating RNA that associates with the crRNA sequence through a region of partial complementarity and serves to recruit a Cas9 nuclease to the protospacer motif. In one embodiment, the tracrRNA is at least 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, or more nucleotides in length. In one embodiment, the tracrRNA is about 85 nucleotides in length.

In one embodiment, the crRNA and tracrRNA are engineered into one polynucleotide sequence referred to herein as a “single guide RNA” or “sgRNA.” The crRNA equivalent portion of the sgRNA is engineered to guide the Cas9 nuclease to target any desired protospacer motif. In one embodiment, the tracrRNA equivalent portion of the sgRNA is engineered to be at least 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, or more nucleotides in length.

It will be understood by the skilled artisan, in light of the present disclosure and the level and nature of skill in the art, how to design, prepare and implement further sgRNA sequences of interest in relation to other diseases or conditions disclosed herein associated with IDPs (aberrant conformational or function of a gene product).

The protospacer motif abuts a short protospacer adjacent motif (PAM), which plays a role in recruiting a Cas9/RNA or Cpf1/RNAcomplex. Cas9 polypeptides recognize PAM motifs specific to the Cas9 polypeptide. Accordingly, the CRISPR/Cas9 system can be used to target and cleave either or both strands of a double-stranded polynucleotide sequence flanked by particular 3′ PAM sequences specific to a particular Cas9 polypeptide. PAMs may be identified using bioinformatics or using experimental approaches. Esvelt et al., 2013, Nature Methods. 10(11):1116-1121, which is hereby incorporated by reference in its entirety.

In one embodiment, a polynucleotide encodes a transiently regulatable Cas9 polypeptide. In one embodiment, the polynucleotide comprises a regulatory element for transient expression of and a polynucleotide encoding a Cas9 polypeptide. A Cas9 polypeptide can be engineered as a double-stranded DNA endonuclease or a nickase or catalytically dead Cas9, and forms a ternary target complex with a crRNA and a tracrRNA for site specific DNA recognition and cleavage if catalytically active. Normally, tracrRNA is involved in the maturation of precursor crRNA. Following co-processing of tracrRNA and pre-crRNA by RNase III, a dual-tracrRNA: crRNA guides the CRISPR-associated endonuclease Cas9 to site-specifically cleave a target DNA, e.g., protospacer sequence.

Unlike Cas9 systems, Cpf1-containing CRISPR-Cas systems have three features. First, Cpf1-associated CRISPR arrays are processed into mature crRNAs without the requirement of an additional trans-activating crRNA (tracrRNA) (Deltcheva et al., 2011, Chylinski et al., 2013). Second, Cpf1-crRNA complexes efficiently cleave target DNA proceeded by a short T-rich protospacer-adjacent motif (5′-TTN PAM), in contrast to the G-rich PAM following the target DNA for Cas9 systems. Third, Cpf1 introduces a staggered DNA double-stranded break with a 4 or 5-nt 5′ overhang.

In one embodiment, a polynucleotide encodes a transiently regulatable Cpf1 polypeptide. In one embodiment, the polynucleotide comprises a regulatory element for transient expression of and a polynucleotide encoding a Cpf1 polypeptide. A Cpf1 polypeptide can be engineered as a double-stranded DNA endonuclease or a nickase or catalytically dead Cpf1, and forms a target complex with a crRNA for site specific DNA recognition and cleavage if catalytically active. Following processing of pre-crRNA by RNase III, a crRNA guides the CRISPR-associated endonuclease Cpf1 to site-specifically cleave a target DNA, e.g., protospacer sequence.

In one embodiment, the one or more crRNAs comprises a pair of offset crRNAs complementary to opposite strands of the target site. In one embodiment, the one or more sgRNAs comprises a pair of offset sgRNAs complementary to opposite strands of the target site. Without wishing to be bound by any particular theory, in some embodiments, it is contemplated that using a pair of offset crRNAs or sgRNAs with a Cas9 or Cpf1 nickase contemplated herein reduces off target genome editing. A single nick is repaired efficiently using a cell's base-excision-repair (BER) machinery. Thus, a large majority of single nicks do not result in nonhomologous end joining (NHEJ)-mediated indels. By inducing offset nicks, off-target single nick events will likely result in very low indel rates.

In one embodiment, offset nicks are induced using a pair of offset crRNAs or sgRNAs with a Cas9 or Cpf1 nickase increases site-specific NHEJ or HDR (when a repair template is provided). In one embodiment, a pair of offset crRNAs or sgRNAs is designed to create 5′ overhangs via the offset nicks to increase the rate of site-specific NHEJ or homologous recombination.

In one embodiment, the pair of offset crRNAs or sgRNAs are offset by at least 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 88, 89, 90, 91, 92, 93, 94, 95, 96, 97, 98, 99, or at least 100 nucleotides.

In one embodiment, the pair of offset crRNAs or sgRNAs are offset by about 5 to about 100 nucleotides, about 10 to about 50 nucleotides, about 10 to about 40 nucleotides, about 10 to about 30 nucleotides, about 10 to about 20 nucleotides, or about 15 to 30 nucleotides, as well as all intermediate lengths or ranges.

In one embodiment, a crRNA or sgRNA is designed to induce a single nick with a Cas9 or Cpf1 nickase; in combination with a double-stranded or single-stranded repair template polynucleotide, the nick is repaired using homologous recombination with minimal off-target indel effects.

Illustrative examples for bacterial sources of Cas9 polynucleotides encoding a Cas9 polypeptide suitable for use in the methods contemplated herein and corresponding PAM motifs include, but are not limited to: Staphylococcus aureus, (NNGRR), Streptococcus pyogenes Cas9 (NGG); Streptococcus thermophilis Cas9 (NNNNGANN, NNNNGTTN, NNNNGNNT, NNAGAAW, NNNNGTNN, NNNNGNTN); Treponema denticola Cas9 (NAAAAN, NAAANC, NANAAC, NNAAAC); and Neisseria meningitidis Cas9 (NNAGAA, NNAGGA, NNGGAA, NNNNGATT, NNANAA, NNGGGA). Without wishing to be bound to any particular theory, a virtually limitless selection of protospacer motifs may be targeted using the CRISPR technology because a suitable Cas9 may be selected to target any protospacer based on the sequence of the adjacent PAM motif.

Illustrative examples for bacterial sources of Cpf1 polynucleotides encoding a Cpf1 polypeptide suitable for use in the methods contemplated herein include, but are not limited to: Francisella novicida, Acidaminococcus sp. BV3L6, and Lachnospiraceae bacterium ND2006.

As used herein, the terms “siRNA” or “short interfering RNA” refer to a short polynucleotide sequence that mediates a process of sequence-specific post-transcriptional gene silencing, translational inhibition, transcriptional inhibition, or epigenetic RNAi in animals (Zamore et al., 2000, Cell, 101, 25-33; Fire et al., 1998, Nature, 391, 806; Hamilton et al., 1999, Science, 286, 950-951; Lin et al., 1999, Nature, 402, 128-129; Sharp, 1999, Genes & Dev., 13, 139-141; and Strauss, 1999, Science, 286, 886). In certain embodiments, an siRNA comprises a first strand and a second strand that have the same number of nucleosides; however, the first and second strands are offset such that the two terminal nucleosides on the first and second strands are not paired with a residue on the complimentary strand. In certain instances, the two nucleosides that are not paired are thymidine resides. The siRNA should include a region of sufficient homology to the target gene, and be of sufficient length in terms of nucleotides, such that the siRNA, or a fragment thereof, can mediate down regulation of the target gene. Thus, an siRNA includes a region which is at least partially complementary to the target RNA. It is not necessary that there be perfect complementarity between the siRNA and the target, but the correspondence must be sufficient to enable the siRNA, or a cleavage product thereof, to direct sequence specific silencing, such as by RNAi cleavage of the target RNA. Complementarity, or degree of homology with the target strand, is most critical in the antisense strand. While perfect complementarity, particularly in the antisense strand, is often desired, some embodiments include one or more, but preferably 10, 8, 6, 5, 4, 3, 2, or fewer mismatches with respect to the target RNA. The mismatches are most tolerated in the terminal regions, and if present are preferably in a terminal region or regions, e.g., within 6, 5, 4, or 3 nucleotides of 5′ and/or 3′ terminus. The sense strand need only be sufficiently complementary with the antisense strand to maintain the overall double-strand character of the molecule. Each strand of an siRNA can be equal to or less than 30, 25, 24, 23, 22, 21, or 20 nucleotides in length. The strand is preferably at least 19 nucleotides in length. For example, each strand can be between 21 and 25 nucleotides in length. Preferred siRNAs have a duplex region of 17, 18, 19, 29, 21, 22, 23, 24, or 25 nucleotide pairs, and one or more overhangs of 2-3 nucleotides, preferably one or two 3′ overhangs, of 2-3 nucleotides.

As used herein, the terms “miRNA” or “microRNA” s refer to small non-coding RNAs of 20-22 nucleotides, typically excised from “70 nucleotide foldback RNA precursor structures known as pre-miRNAs. miRNAs negatively regulate their targets in one of two ways depending on the degree of complementarity between the miRNA and the target. First, miRNAs that bind with perfect or nearly perfect complementarity to protein-coding mRNA sequences induce the RNA-mediated interference (RNAi) pathway. miRNAs that exert their regulatory effects by binding to imperfect complementary sites within 3′ untranslated regions (UTRs) of their mRNA targets, repress target-gene expression post-transcriptionally, apparently at the level of translation, through a RISC complex that is similar to, or possibly identical with, the one that is used for the RNAi pathway. Consistent with translational control, miRNAs that use this mechanism reduce the protein levels of their target genes, but the mRNA levels of these genes are only minimally affected. miRNAs encompass both naturally occurring miRNAs as well as artificially designed miRNAs that can specifically target any mRNA sequence. For example, in one embodiment, the skilled artisan can design short hairpin RNA constructs expressed as human miRNA (e.g., miR-30 or miR-21) primary transcripts or “mishRNA.” This design adds a Drosha processing site to the hairpin construct and has been shown to greatly increase knockdown efficiency (Pusch et al., 2004). The hairpin stem consists of 22-nt of dsRNA (e.g., antisense has perfect complementarity to desired target) and a 15-19-nt loop from a human miR. Adding the miR loop and miR30 flanking sequences on either or both sides of the hairpin results in greater than 10-fold increase in Drosha and Dicer processing of the expressed hairpins when compared with conventional shRNA designs without microRNA. Increased Drosha and Dicer processing translates into greater siRNA/miRNA production and greater potency for expressed hairpins.

In one embodiment, a polynucleotide encoding a CRISPR-Cas endonuclease comprises an intron that comprises a miRNA and a 3′UTR that comprises a corresponding miRNA target site. Without wishing to be bound to any particular theory, it is contemplated that this architecture can be used to transiently regulate the expression of the CRISPR-Cas endonuclease and minimize the off-target effects of the endonuclease either alone or in combination with one or more additional regulatory elements to regulate the transient expression of the endonuclease.

As used herein, the terms “shRNA” or “short hairpin RNA” refer to double-stranded structure that is formed by a single self-complementary RNA strand. shRNA constructs containing a nucleotide sequence identical to a portion, of either coding or non-coding sequence, of the target gene are preferred for inhibition. RNA sequences with insertions, deletions, and single point mutations relative to the target sequence have also been found to be effective for inhibition. Greater than 90% sequence identity, or even 100% sequence identity, between the inhibitory RNA and the portion of the target gene is preferred. In certain preferred embodiments, the length of the duplex-forming portion of an shRNA is at least 20, 21 or 22 nucleotides in length, e.g., corresponding in size to RNA products produced by Dicer-dependent cleavage. In certain embodiments, the shRNA construct is at least 25, 50, 100, 200, 300 or 400 bases in length. In certain embodiments, the shRNA construct is 400-800 bases in length. shRNA constructs are highly tolerant of variation in loop sequence and loop size.

As used herein, the term “ribozyme” refers to a catalytically active RNA molecule capable of site-specific cleavage of target mRNA. Several subtypes have been described, e.g., hammerhead and hairpin ribozymes. Ribozyme catalytic activity and stability can be improved by substituting deoxyribonucleotides for ribonucleotides at noncatalytic bases. While ribozymes that cleave mRNA at site-specific recognition sequences can be used to destroy particular mRNAs, the use of hammerhead ribozymes is preferred. Hammerhead ribozymes cleave mRNAs at locations dictated by flanking regions that form complementary base pairs with the target mRNA. The sole requirement is that the target mRNA has the following sequence of two bases: 5′-UG-3′. The construction and production of hammerhead ribozymes is well known in the art.

In one embodiment, an expression cassette comprises one or more of a crRNA, a tracrRNA, sgRNA, an siRNA, an miRNA, an shRNA, or a ribozyme and further comprises one or more regulatory sequences, such as, for example, a strong constitutive RNA pol III promoter, e.g., human or mouse U6 snRNA promoter, the human and mouse H1 RNA promoter, or the human tRNA-val promoter; an inducible RNA pol III promoter, e.g., U6-6TetO promoter, H1-peroxide promoter; or a strong constitutive or inducible RNA pol II promoter, as described elsewhere herein.

The polynucleotides contemplated herein, regardless of the length of the coding sequence itself, may be combined with other DNA sequences, such as expression control sequences, regulatory elements, promoters and/or enhancers, untranslated regions (UTRs), Kozak sequences, polyadenylation signals, additional restriction enzyme sites, multiple cloning sites, internal ribosomal entry sites (IRES), recombinase recognition sites (e.g., LoxP, FRT, and Att sites), guide RNA target sites, termination codons, transcriptional termination signals, and polynucleotides encoding self-cleaving polypeptides, epitope tags, as disclosed elsewhere herein or as known in the art, such that their overall length may vary considerably. It is therefore contemplated that a polynucleotide fragment of almost any length may be employed, with the total length preferably being limited by the ease of preparation and use in the intended recombinant DNA protocol.

Polynucleotides can be prepared, manipulated and/or expressed using any of a variety of well established techniques known and available in the art. In order to express a desired polypeptide, a nucleotide sequence encoding the polypeptide, can be inserted into an appropriate vector, such as a viral vector. Illustrative examples of viral vectors suitable for use in particular embodiments include, but are not limited to lentiviral vectors, adenovirus vectors, and adeno-associated virus (AAV) vectors. In preferred embodiments, the viral vector is an AAV vector.

“Expression control sequences,” “control elements,” or “regulatory sequences” present in an expression vector are those non-translated regions of the vector-origin of replication, selection cassettes, promoters, enhancers, translation initiation signals (Shine Dalgarno sequence or Kozak sequence) introns, a polyadenylation sequence, 5′ and 3′ untranslated regions-which interact with host cellular proteins to carry out transcription and translation. Such elements may vary in their strength and specificity. Depending on the vector system and host utilized, any number of suitable transcription and translation elements, including ubiquitous promoters and inducible promoters may be used.

In particular embodiments, a polynucleotide for use in practicing the invention is a vector, including but not limited to expression vectors and viral vectors, and includes exogenous, endogenous, or heterologous control sequences such as promoters and/or enhancers. An “endogenous” control sequence is one which is naturally linked with a given gene in the genome. An “exogenous” control sequence is one which is placed in juxtaposition to a gene by means of genetic manipulation (i.e., molecular biological techniques) such that transcription of that gene is directed by the linked enhancer/promoter. A “heterologous” control sequence is an exogenous sequence that is from a different species than the cell being genetically manipulated.

The term “promoter” as used herein refers to a recognition site of a polynucleotide (DNA or RNA) to which an RNA polymerase binds. An RNA polymerase initiates and transcribes polynucleotides operably linked to the promoter. In particular embodiments, promoters operative in mammalian cells comprise an AT-rich region located approximately 25 to 30 bases upstream from the site where transcription is initiated and/or another sequence found 70 to 80 bases upstream from the start of transcription, a CNCAAT region where N may be any nucleotide. In particular embodiments, the vector comprises one or more RNA pol II and/or RNA pol III promoters.

Illustrative examples of RNA pol II promoters suitable for use in particular embodiments include, but are not limited to a neuron specific promoter.

The term “enhancer” refers to a segment of DNA which contains sequences capable of providing enhanced transcription and in some instances can function independent of their orientation relative to another control sequence. An enhancer can function cooperatively or additively with promoters and/or other enhancer elements. The term “promoter/enhancer” refers to a segment of DNA which contains sequences capable of providing both promoter and enhancer functions.

The term “operably linked”, refers to a juxtaposition wherein the components described are in a relationship permitting them to function in their intended manner. In one embodiment, the term refers to a functional linkage between a nucleic acid expression control sequence (such as a promoter, and/or enhancer) or regulatory element and a second polynucleotide sequence, e.g., a polynucleotide-of-interest, wherein the expression control sequence or regulatory element directs transcription of the nucleic acid corresponding to the second sequence.

As used herein, the term “constitutive expression control sequence” refers to a promoter, enhancer, or promoter/enhancer that continually or continuously allows for transcription of an operably linked sequence. A constitutive expression control sequence may be a “ubiquitous” promoter, enhancer, or promoter/enhancer that allows expression in a wide variety of cell and tissue types or a “cell specific,” “cell type specific,” “cell lineage specific,” or “tissue specific” promoter, enhancer, or promoter/enhancer that allows expression in a restricted variety of cell and tissue types, respectively.

Illustrative ubiquitous expression control sequences suitable for use in particular embodiments of the invention include, but are not limited to, a cytomegalovirus (CMV) immediate early promoter, a viral simian virus 40 (SV40) (e.g., early or late), a Moloney murine leukemia virus (MoMLV) LTR promoter, a Rous sarcoma virus (RSV) LTR, a herpes simplex virus (HSV) (thymidine kinase) promoter, H5, P7.5, and P11 promoters from vaccinia virus, an elongation factor 1-alpha (EF1a) promoter, early growth response 1 (EGR1), ferritin H (FerH), ferritin L (FerL), Glyceraldehyde 3-phosphate dehydrogenase (GAPDH), eukaryotic translation initiation factor 4A1 (EIF4A1), heat shock 70 kDa protein 5 (HSPA5), heat shock protein 90 kDa beta, member 1 (HSP90B1), heat shock protein 70 kDa (HSP70), β-kinesin (β-KIN), the human ROSA 26 locus (Irions et al., Nature Biotechnology 25, 1477-1482 (2007)), a Ubiquitin C promoter (UBC), a phosphoglycerate kinase-1 (PGK) promoter, and a cytomegalovirus enhancer/chicken β-actin (CAG) promoter.

In a particular embodiments, it may be desirable to use a tissue-specific promoter to achieve cell-type specific, lineage specific, or tissue-specific expression of a desired polynucleotide sequence. Any of a wide variety of tissue-specific promoters are known to those skilled in the art with respect to cell and tissue types of interest. For example, in certain embodiments, illustrative tissue-specific promoters include, but are not limited to: a glial fibrillary acidic protein (GFAP) promoter (astrocyte expression), a synapsin promoter (neuron expression), and calcium/calmodulin-dependent protein kinase II (neuron expression), tubulin alpha I (neuron expression), neuron-specific enolase (neuron expression), platelet-derived growth factor beta chain (neuron expression), a TRPV1 promoter (neuron expression), a Nav1.7 promoter (neuron expression), a Nav1.8 promoter (neuron expression), a Nav1.9 promoter (neuron expression), or an Advillin promoter (neuron expression).

According to certain embodiments, the cell type specific promoter is specific for cell types found in the brain (e.g., neurons, glial cells).

As used herein, “conditional expression” may refer to any type of conditional expression including, but not limited to, inducible expression; repressible expression; expression in cells or tissues having a particular physiological, biological, or disease state, etc. This definition is not intended to exclude cell type or tissue specific expression. Certain embodiments of the invention provide conditional expression of a polynucleotide-of-interest, e.g., expression is controlled by subjecting a cell, tissue, organism, etc., to a treatment or condition that causes the polynucleotide to be expressed or that causes an increase or decrease in expression of the polynucleotide encoded by the polynucleotide-of-interest.

Illustrative examples of inducible promoters/systems include, but are not limited to, steroid-inducible promoters such as promoters for genes encoding glucocorticoid or estrogen receptors (inducible by treatment with the corresponding hormone), metallothionine promoter (inducible by treatment with various heavy metals), MX-1 promoter (inducible by interferon), the “GeneSwitch” mifepristone-regulatable system (Sirin et al., 2003, Gene, 323:67), the cumate inducible gene switch (WO 2002/088346), tetracycline-dependent regulatory systems, etc.

Illustrative examples of promoters suitable for use in particular embodiments include, but are not limited to neuron specific promoters.

In particular embodiments, a polynucleotide contemplated herein comprises a neuron specific promoter or a promoter operative in a neuronal cell.

In particular embodiments, a polynucleotide contemplated herein comprises a neuron specific promoter operable in a trigeminal ganglion (TGG) neuron or a dorsal root ganglion (DRG) neuron.

In particular embodiments, a polynucleotide contemplated herein comprises a neuron specific promoter selected from the group consisting of a calcium/calmodulin-dependent protein kinase II promoter, a tubulin alpha I promoter, a neuron-specific enolase promoter, a platelet-derived growth factor beta chain promoter, an hSYN1 promoter, a TRPV1 promoter, a Nav1.7 promoter, a Nav1.8 promoter, a Nav1.9 promoter, and an Advillin promoter.

In one embodiment, the neuron specific promoter is a human synapsin 1 (SYN1) promoter.

In particular embodiments, polynucleotides contemplated herein comprise at least one (typically two) site(s) for recombination mediated by a site specific recombinase. As used herein, the terms “recombinase” or “site specific recombinase” include excusive or integrative proteins, enzymes, co-factors or associated proteins that are involved in recombination reactions involving one or more recombination sites (e.g., two, three, four, five, six, seven, eight, nine, ten or more.), which may be wild-type proteins (see Landy, Current Opinion in Biotechnology 3:699-707 (1993)), or mutants, derivatives (e.g., fusion proteins containing the recombination protein sequences or fragments thereof), fragments, and variants thereof. Illustrative examples of recombinases suitable for use in particular embodiments of the present invention include, but are not limited to: Cre, Int, IHF, Xis, Flp, Fis, Hin, Gin, ΦC31, Cin, Tn3 resolvase, TndX, XerC, XerD, TnpX, Hjc, Gin, SpCCE1, and ParA.

The polynucleotides may comprise one or more recombination sites for any of a wide variety of site specific recombinases. As used herein, the terms “recombination sequence,” “recombination site,” or “site specific recombination site” refer to a particular nucleic acid sequence to which a recombinase recognizes and binds.

For example, one recombination site for Cre recombinase is loxP which is a 34 base pair sequence comprising two 13 base pair inverted repeats (serving as the recombinase binding sites) flanking an 8 base pair core sequence (see FIG. 1 of Sauer, B., Current Opinion in Biotechnology 5:521-527 (1994)). Other exemplary loxP sites include, but are not limited to: lox511 (Hoess et al., 1996; Bethke and Sauer, 1997), lox5171 (Lee and Saito, 1998), lox2272 (Lee and Saito, 1998), m2 (Langer et al., 2002), lox71 (Albert et al., 1995), and lox66 (Albert et al., 1995).

Suitable recognition sites for the FLP recombinase include, but are not limited to: FRT (McLeod, et al., 1996), F₁, F₂, F₃ (Schlake and Bode, 1994), F₄, F₅ (Schlake and Bode, 1994), FRT(LE) (Senecoff et al., 1988), FRT(RE) (Senecoff et al., 1988).

Other examples of recognition sequences are the attB, attP, attL, and attR sequences, which are recognized by the recombinase enzyme λ Integrase, e.g., phi-c31. The φC31 SSR mediates recombination only between the heterotypic sites attB (34 bp in length) and attP (39 bp in length) (Groth et al., 2000). attB and attP, named for the attachment sites for the phage integrase on the bacterial and phage genomes, respectively, both contain imperfect inverted repeats that are likely bound by φC31 homodimers (Groth et al., 2000). The product sites, attL and attR, are effectively inert to further C31-mediated recombination (Belteki et al., 2003), making the reaction irreversible. For catalyzing insertions, it has been found that attB-bearing DNA inserts into a genomic attP site more readily than an attP site into a genomic attB site (Thyagarajan et al., 2001; Belteki et al., 2003). Thus, typical strategies position by homologous recombination an attP-bearing “docking site” into a defined locus, which is then partnered with an attB-bearing incoming sequence for insertion.

In particular embodiments, polynucleotides contemplated herein, include one or more polynucleotides-of-interest that encode one or more polypeptides. In particular embodiments, to achieve efficient translation of each of the plurality of polypeptides, the polynucleotide sequences can be separated by one or more IRES sequences or polynucleotide sequences encoding self-cleaving polypeptides.

As used herein, an “internal ribosome entry site” or “IRES” refers to an element that promotes direct internal ribosome entry to the initiation codon, such as ATG, of a cistron (a protein encoding region), thereby leading to the cap-independent translation of the gene. See, e.g., Jackson et al., 1990. Trends Biochem Sci 15(12):477-83) and Jackson and Kaminski. 1995. RNA 1(10):985-1000. Examples of IRES generally employed by those of skill in the art include those described in U.S. Pat. No. 6,692,736. Further examples of “IRES” known in the art include, but are not limited to IRES obtainable from picornavirus (Jackson et al., 1990) and IRES obtainable from viral or cellular mRNA sources, such as for example, immunoglobulin heavy-chain binding protein (BiP), the vascular endothelial growth factor (VEGF) (Huez et al. 1998. Mol. Cell. Biol. 18(11):6178-6190), the fibroblast growth factor 2 (FGF-2), and insulin-like growth factor (IGFII), the translational initiation factor elF4G and yeast transcription factors TFIID and HAP4, the encephelomycarditis virus (EMCV) which is commercially available from Novagen (Duke et al., 1992. J. Virol 66(3):1602-9) and the VEGF IRES (Huez et al., 1998. Mol Cell Biol 18(11):6178-90). IRES have also been reported in viral genomes of Picornaviridae, Dicistroviridae and Flaviviridae species and in HCV, Friend murine leukemia virus (FrMLV) and Moloney murine leukemia virus (MoMLV).

In one embodiment, the IRES used in polynucleotides contemplated herein is an EMCV IRES.

In particular embodiments, a polynucleotide encoding a polypeptide comprises a consensus Kozak sequence. As used herein, the term “Kozak sequence” refers to a short nucleotide sequence that greatly facilitates the initial binding of mRNA to the small subunit of the ribosome and increases translation. The consensus Kozak sequence is (GCC)RCCATGG (SEQ ID NO:56), where R is a purine (A or G) (Kozak, 1986. Cell. 44(2):283-92, and Kozak, 1987. Nucleic Acids Res. 15(20):8125-48).

In particular embodiments, polynucleotides comprise a polyadenylation sequence 3′ of a polynucleotide encoding a polypeptide to be expressed. Polyadenylation sequences can promote mRNA stability by addition of a polyA tail to the 3′ end of the coding sequence and thus, contribute to increased translational efficiency. Cleavage and polyadenylation is directed by a poly(A) sequence in the RNA. The core poly(A) sequence for mammalian pre-mRNAs has two recognition elements flanking a cleavage-polyadenylation site. Typically, an almost invariant AAUAAA hexamer lies 20-50 nucleotides upstream of a more variable element rich in U or GU residues. Cleavage of the nascent transcript occurs between these two elements and is coupled to the addition of up to 250 adenosines to 5′ cleavage product. In particular embodiments, the core poly(A) sequence is an ideal polyA sequence (e.g., AATAAA, ATTAAA, AGTAAA). In particular embodiments the poly(A) sequence is an SV40 polyA sequence, a bovine growth hormone polyA sequence (BGHpA), a rabbit β-globin polyA sequence (rβgpA), or another suitable heterologous or endogenous polyA sequence known in the art.

### REFERENCES

Sickmeier, M. et al. DisProt: the Database of Disordered Proteins. Nucleic Acids Research 35, D786-D793 (2007)

What is claimed is:

1. A method to diagnose an effect on a subject of a mutation in an intrinsically disordered protein or an intrinsically disordered region thereof, comprising:
determining a mutation from a known amino acid sequence of an intrinsically disordered protein or an intrinsically disordered region thereof, the known amino acid sequence associated with a known value of gyration radius [symbol] R_{g} [symbol] or end to end distance [symbol] R_{N} [symbol] ;
determining values of a plurality of physical properties of the mutation based on the mutation in the amino acid sequence input to a polymer physics-based model;
determining a quick value of [symbol] R_{g} [symbol] or [symbol] R_{N} [symbol] or both of the mutation based on output produced by inputting the values of the plurality of physical properties of the mutation to a neural network trained on a training set including multiple instances of training set values for [symbol] R_{g} [symbol] or [symbol] R_{N} [symbol] or both with corresponding training set values of the plurality of physical properties; and
using a difference between the quick value and the known value to determine an effect of the mutation.

2. The method as recited in claim 1 wherein the neural network comprises a plurality of fully connected hidden layers.

3. The method as recited in claim 2 wherein the neural network comprises six fully connected hidden layers.

4. The method as recited in claim 2 wherein each hidden layer is configured to drop a number of nodes by a factor of two or three.

5. The method as recited in claim 2 wherein alternate hidden layers alternate between a tanh activation function for the hidden layer and a RELU activation function for the hidden layer.

6. The method as recited in claim 2 wherein the first hidden layer following the input layer comprises 192 nodes.

7. The method as recited in claim 2 wherein the last hidden layer before the output layer uses a linear activation function.

8. The method as recited in claim 1 further comprising performing detailed modeling to determine improved magnitude of gyration of mutations that have an effect greater than a first threshold.

9. The method as recited in claim 8 further comprising performing drug screening for pathogenic mutations that have an improved magnitude greater than a second threshold.

10. The method as recited in claim 1, wherein the plurality of physical properties includes five or more of a group including length (N), center of mass CM(m), center of the charge CM(q), center of hydropathy CM(λ), mass mean field standard deviation (MFSTD(m)), charge MFSTD(q), hydropathy MFSTD(λ), entropy, charge entropy, net charge par residue (qnet=Q/N), net positive charge par residue q^{+ve}/N, net half positive charge par residue q^{+1/2ve}/N, net negative charge par residue q^{-ve}/N, net neutral charge par residue q⁰/N, charge asymmetry ƒ*, charge decoration parameter (SCD), hydropathy asymmetry (<λ>), contiguous patches of unit positive charge (Pq^{+ve}), contiguous patches of unit negative charge (Pq^{-ve}), contiguous patches of half positive charge (Pq^{1/2ve}), and contiguous patches of neutral charge (Pq⁰).

11. A non-transitory computer readable medium configured to diagnose an effect on a subject of a mutation in an intrinsically disordered protein or an intrinsically disordered region thereof, the non-transitory computer readable medium carrying one or more sequences of instructions, wherein execution of the one or more sequences of instructions by one or more processors causes the one or more processors to perform the steps of:
determining a mutation from a known amino acid sequence of an intrinsically disordered protein or an intrinsically disordered region thereof, the known amino acid sequence associated with a known value of gyration radius [symbol] R_{g} [symbol] or end to end distance [symbol] R_{N} [symbol] ;
determining values of a plurality of physical properties of the mutation based on the mutation in the amino acid sequence input to a polymer physics-based model;
determining a quick value of [symbol] R_{g} [symbol] or [symbol] R_{N} [symbol] or both of the mutation based on output produced by inputting the values of the plurality of physical properties of the mutation to a neural network trained on a training set including multiple instances of training set values for [symbol] R_{g} [symbol] or [symbol] R_{N} [symbol] or both with corresponding training set values of the plurality of physical properties; and
using a difference between the quick value and the known value to determine an effect of the mutation.

12. An apparatus configured to diagnose an effect on a subject of a mutation in an intrinsically disordered protein or an intrinsically disordered region thereof, the apparatus comprising:
at least one processor; and
at least one memory including one or more sequences of instructions, the at least one memory and the one or more sequences of instructions configured to, with the at least one processor, cause the apparatus to perform at least the steps of
determining a mutation from a known amino acid sequence of an intrinsically disordered protein or an intrinsically disordered region thereof, the known amino acid sequence associated with a known value of gyration radius [symbol] R_{g} [symbol] or end to end distance [symbol] R_{N} [symbol] ;
determining values of a plurality of physical properties of the mutation based on the mutation in the amino acid sequence input to a polymer physics-based model;
determining a quick value of [symbol] R_{g} [symbol] or [symbol] R_{N} [symbol] or both of the mutation based on output produced by inputting the values of the plurality of physical properties of the mutation to a neural network trained on a training set including multiple instances of training set values for [symbol] R_{g} [symbol] or [symbol] R_{N} [symbol] or both with corresponding training set values of the plurality of physical properties; and
using a difference between the quick value and the known value to determine an effect of the mutation.
